(19) United States
(12) Patent Application Publication
(10) Pub. No.: US 2004/0088744 A1
(43) Pub. Date: May 6, 2004

(54) Fatty liver disease resistant bovines

(76) Inventors: Alan Attie, Madison, WI (US); Gregory Bleck, Cross Plains, WI (US); Robert Bremel, Hillpoint, WI (US); Jane Homan, Hillpoint, WI (US)

Correspondence Address: J Mitchell Jones, Medlen & Carroll, Suite 350, 101 Howard Street, San Francisco, CA, 94105, US

(21) Appl. No.: 10/344,115

(22) PCT Filed: Aug. 10, 2001
(86) PCT No.: PCT/US01/25235

(51) Int. Cl. A01K 67/027

(52) U.S. Cl. 800/15

(57) ABSTRACT

The present invention provides methods for the production of a transgenic bovine. In particular, the present invention provides methods for generating transgenic bovines with transgenes that enhance the ability of the bovines to metabolize lipids. The present invention thus provides bovines resistant to fatty liver disease. The compositions and methods of the present invention provide a solution to costly disease.

### FIELD OF THE INVENTION

The present invention provides methods for the production of transgenic bovines. In particular, the present invention provides methods for generating transgenic bovines with transgenes that enhance the ability of the bovines to metabolize lipids. The present invention thus provides bovines resistant to fatty liver disease.

### BACKGROUND OF THE INVENTION

Dairy cows are vulnerable to the accumulation of lipids in the liver. During pregnancy and shortly after parturition, 25-60% of dairy cows suffer from moderate to severe fatty liver degeneration. Fatty liver degeneration gives rise to a well-recognized disease syndrome, including increased susceptibility to opportunistic infectious diseases of the udder and uterus. Cows with fatty liver loose their appetite, have impaired urea metabolism, and appear to be more vulnerable to ketosis. Cows with fatty liver tend to have reduced fertility and increased risk of displaced abomasum, metritis, and retained placenta. A coincident reduced vitamin D metabolism increases the risk of nonresponding hypocalcinemia, or milk fever. Infectious diseases resulting from fatty liver disease are treated with antibiotics, driving the usage of large amounts of antibiotics in dairy herds.

Ketosis is one common complication associated with fatty liver disease in bovines. Ketosis most often occurs in high producing cattle. Ketosis is estimated to occur in 8-12% of all US dairy cows (some 900 thousand cases per year) and is estimated to cost approximately $140 in treatment and lost production for each occurrence (Hoard's Dairyman, [1996]). This represents a little over $128 million dollars each year in the United States. Using the value of dairy products as the yard stick, ketosis reduces the value of all dairy products by approximately 0.7%. Even if affected cows survive, fatty liver degeneration leads to reduced milk production and additional economic losses to the dairy industry.

There are no known methods of preventing fatty liver disease and the associated complications. Thus, the art is in need of a reliable, cost effective method of reducing the incidence of fatty liver disease in bovines.

### SUMMARY OF THE INVENTION

The present invention provides methods for the production of a transgenic bovine. In particular, the present invention provides methods for generating transgenic bovines with transgenes that enhance the ability of the bovines to metabolize lipids. The present invention thus provides bovines resistant to fatty liver disease.

In some embodiments, the present invention provides nucleic acid constructs comprising a liver specific promoter operably linked to a gene of interest selected from the group consisting of ApoE and truncated soluble LDL receptor. The present invention is not limited to any particular gene encoding ApoE. Indeed, the use of a variety of mutant, variant and homologous ApoE gene sequences is contemplated, including, but not limited to ApoE gene sequences selected from the group consisting of SEQ ID NO:15 and sequences hybridizable to SEQ ID NO:15 under conditions of low to high stringency. The present invention is not limited to the use any particular truncated soluble LDL receptor gene sequence. Indeed, the use of a variety of mutant, variant and homologous soluble LDL receptor gene sequences is contemplated, including but not limited to truncated soluble LDL receptor gene sequences selected from the group consisting of SEQ ID NO:16 and sequences hybridizable to SEQ ID NO:16 under conditions of low to high stringency. The present invention is not limited to the use of any particular liver-specific promoter. Indeed, the use of a variety of mutant, variant and homologous liver-specific promoters is contemplated, including, but not limited to the bovine alpha-1-antitrypsin and albumin promoters. The present invention is not limited to any particular sequence encoding the bovine alpha-1-antitrypsin promoter. Indeed, the use of a variety of promoter sequences is contemplated, including, but not limited to sequences encoded by SEQ ID NO:14 and sequences hybridizable to SEQ ID NO: 14 under conditions of low to high stringency. The present invention is not limited to any particular sequence encoding the bovine albumin promoter. Indeed, the use of a variety of promoter sequences is contemplated, including, but not limited to sequences encoded by SEQ ID NO:13 and sequences hybridizable to SEQ ID NO: 13 under conditions of low to high stringency. In particularly preferred embodiments, the constructs further comprise retroviral elements including, but not limited to, retroviral 3′ and 5′ LTRs. In further preferred embodiments, the present invention provides transgenic bovines comprising any of the constructs described above.

The present invention also provides methods and processes for producing fatty liver disease resistant transgenic bovines, comprising providing a vector comprising a liver-specific promoter operably linked to a gene of interest selected from ApoE (e.g., a bovine ApoE gene) and truncated soluble LDL receptor (e.g., a bovine truncated soluble LDL receptor gene), a cell selected from oocytes and zygotes and a bovine; introducing the vector into the cell; and transplanting the cell into the bovine to generate a transgenic bovine, wherein the transgenic bovine has increased lipid mobility as compared to a non-transgenic bovine.

In some embodiments, the cell is an oocyte. In other embodiments, the vector is a retroviral vector. As described above, the present invention is not limited to any particular liver-specific promoter. In some preferred embodiments, the promoter is a bovine albumin promoter. In other preferred embodiments, the bovine albumin promoter is SEQ ID NO:13 or sequences hybridizable to SEQ ID NO:13 under conditions of low to high stringency. In other preferred embodiments, the promoter is a bovine alpha-1-antitrypsin promoter. In some embodiments, the bovine alpha-1-antitrypsin promoter is SEQ ID NO:14 or sequences hybridizable to SEQ ID NO:14 under conditions of low to high stringency.

As described above, the present invention is not limited to any particular ApoE gene sequence. In some embodiments, the bovine ApoE gene is SEQ ID NO:15; while in other embodiments, the ApoE gene comprises a sequence hybridizable to SEQ ID NO:15 under conditions of low to high stringency. Likewise, as described above, the present invention is not limited to any particular truncated soluble LDL receptor gene sequence. In some embodiments, the bovine truncated soluble LDL receptor gene comprises SEQ ID NO:16 or sequences hybridizable to SEQ ID NO:16 under conditions of low to high stringency.

The present invention further provides a transgenic bovine produced by the method described above. In some embodiments, the transgenic bovine has increased resistance to fatty liver disease as compared to a non-transgenic bovine. In other embodiments, the transgenic bovine has an increased level of apoB protein secretion as compared to a non-transgenic bovine. In still further embodiments, the transgenic bovine has increased lipid mobility as compared to a non-transgenic bovine.

The present invention also provides a fatty liver disease resistant bovine comprising a transgene comprising a ApoE gene (e.g., a bovine ApoE gene) under the control of a liver-specific promoter selected from the group consisting of an alpha-1-antitrypsin promoter (e.g., bovine alpha-1-antitrypsin promoter) and an albumin promoter (e.g. bovine albumin promoter). In some embodiments, the transgenic bovine has increased resistance to fatty liver disease as compared to a non-transgenic bovine. Additionally, in some embodiments, the transgenic bovine has an increased level of apoB protein secretion as compared to a non-transgenic bovine. In some embodiments, the transgenic bovine has increased lipid mobility as compared to a non-transgenic bovine. In some embodiments, the ApoE gene comprises SEQ ID NO:15; while in other embodiments, the ApoE gene comprises a sequence hybridizable to SEQ ID NO:15 under conditions of low to high stringency.

The present invention further provides a fatty liver disease resistant bovine comprising a transgene comprising a truncated soluble LDL receptor gene under the control of a liver-specific promoter selected from the group consisting of an alpha-1-antitrypsin promoter and an albumin promoter. In some embodiments, the transgenic bovine has increased resistance to fatty liver disease as compared to a non-transgenic bovine. Additionally, in some embodiments, the transgenic bovine has an increased level of apoB protein secretion as compared to a non-transgenic bovine. In some embodiments, the transgenic bovine has increased lipid mobility as compared to a non-transgenic bovine. In some embodiments, the truncated soluble LDL receptor gene comprises SEQ ID NO:16 or sequences hybridizable to SEQ ID NO:16 under conditions of low to high stringency.

In still further embodiments, the present invention provides methods and processes for modifying the metabolism of bovines. Accordingly, in some embodiments, the present invention provides methods comprising providing a bovine oocyte, zygote, or embryo, and an exogenous gene construct encoding a gene that regulates metabolism, and transfecting or transducing the oocyte, zygote, or embryo with the exogenous gene construct so that the metabolism of the resulting transgenic animal is altered as compared to non-transgenic animals. The present invention is not limited to any particular method of creating transgenic animals. Indeed, a variety of methods may be utilized, including, but not limited to retroviral infection of oocytes, retroviral infection of zygotes or embryos, nuclear transfer with genetically modified donor cells, and pronuclear injection. The present invention is not limited to any particular exogenous gene construct. Indeed, a variety of constructs are contemplated, including, but not limited to retroviral vectors and other expression vectors. In some particularly preferred embodiments, the exogenous gene is in operable combination with a tissue specific promoter (e.g., a liver specific promoter). In other embodiments, the exogenous gene is in operable combination with a constitutive promoter (e.g., CMV promoter). In some embodiments, the promoter is a non-mammary specific promoter.

In further embodiments, the present invention encompasses any composition or method as substantially described herein in any of the claims or examples.

### DESCRIPTION OF THE FIGURES

FIG. 1 shows the nucleic acid sequence of SEQ ID NO: 9

FIG. 2 shows the nucleic acid sequence of SEQ ID NO: 10

FIG. 3 shows the nucleic acid sequence of SEQ ID NO: 11

FIG. 4 shows the nucleic acid sequence of SEQ ID NO: 12

FIG. 5 shows the nucleic acid sequence of SEQ ID NO: 13

FIG. 6 shows the nucleic acid sequence of SEQ ID NO: 14

FIG. 7 shows the nucleic acid sequence of SEQ ID NO: 15

FIG. 8 shows the nucleic acid sequence of SEQ ID NO: 16

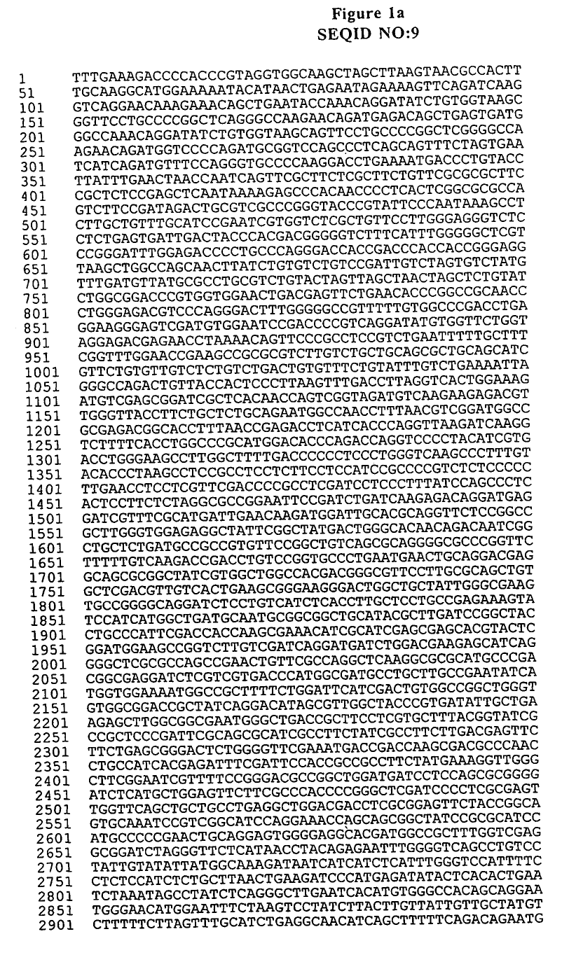

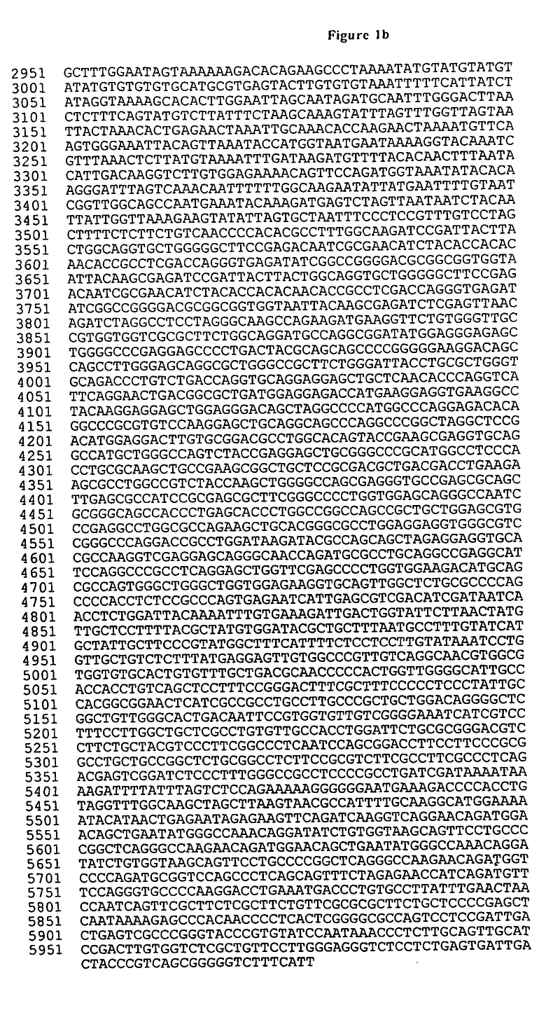

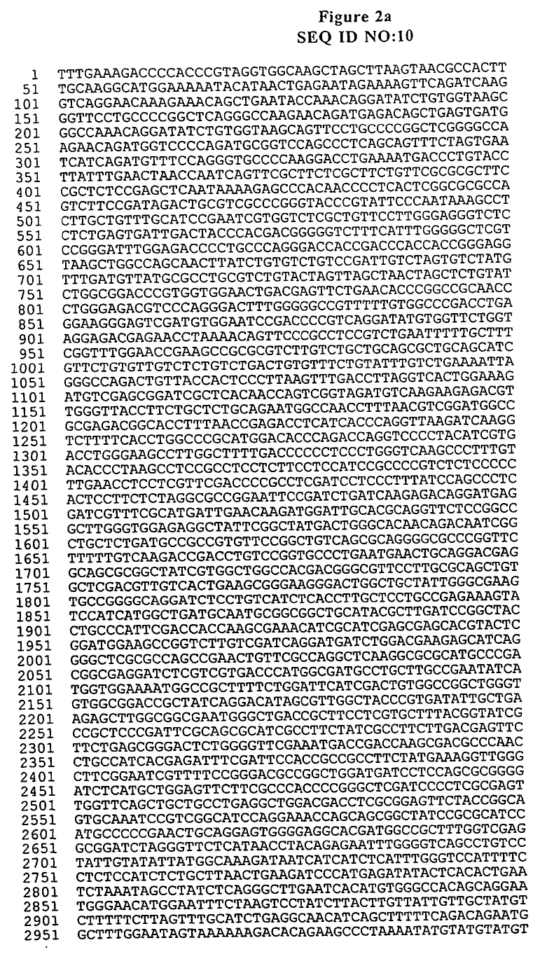

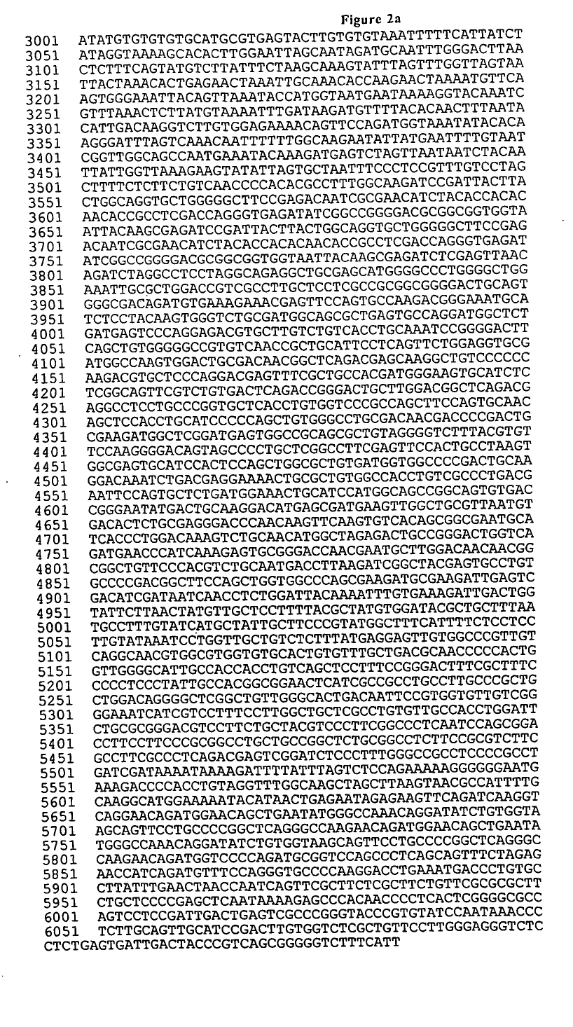

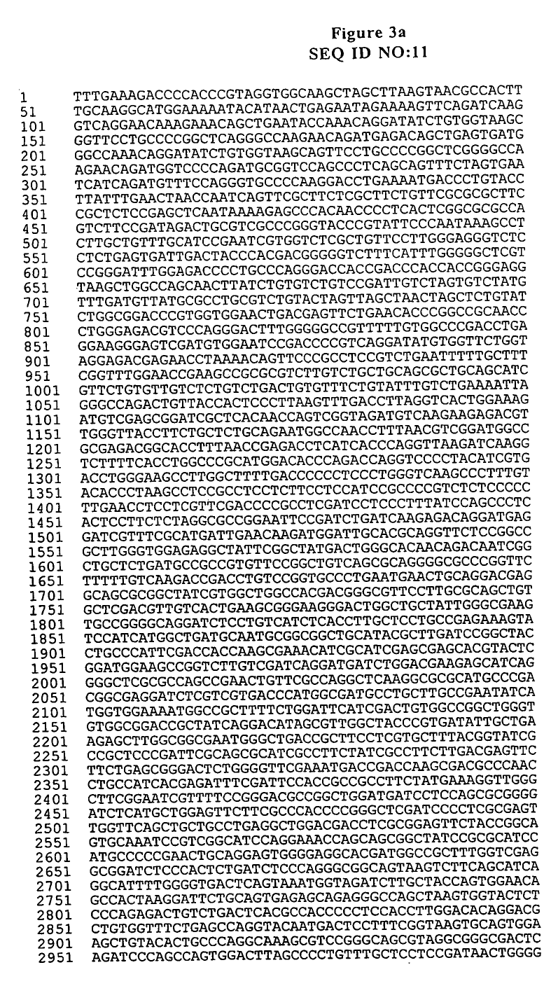

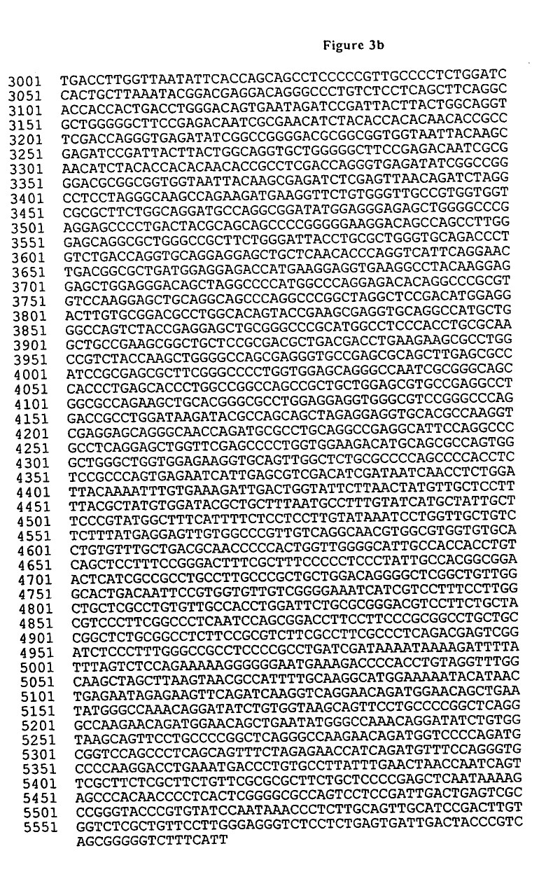

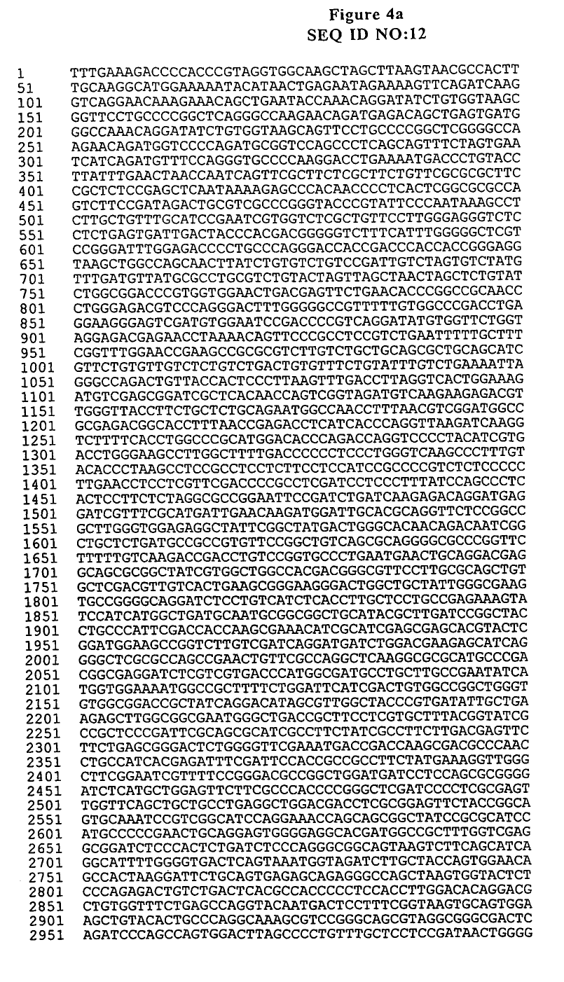

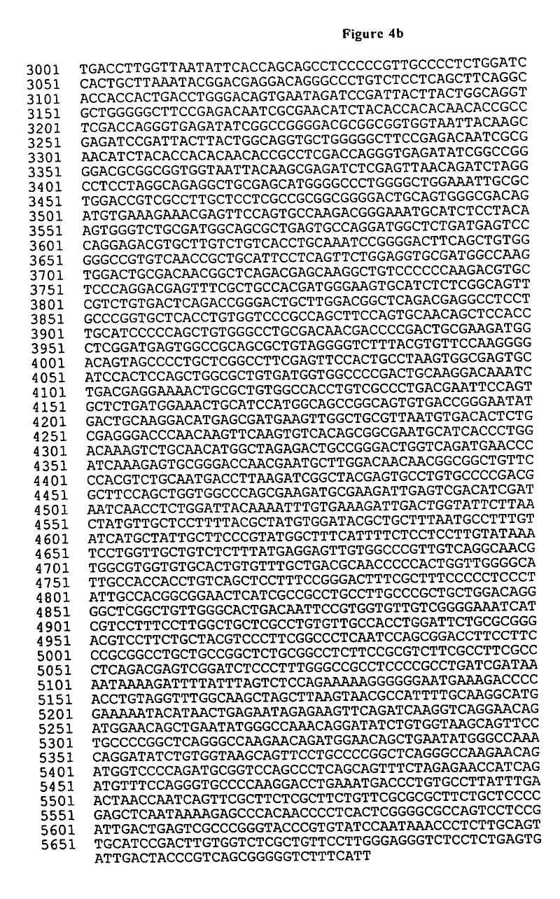

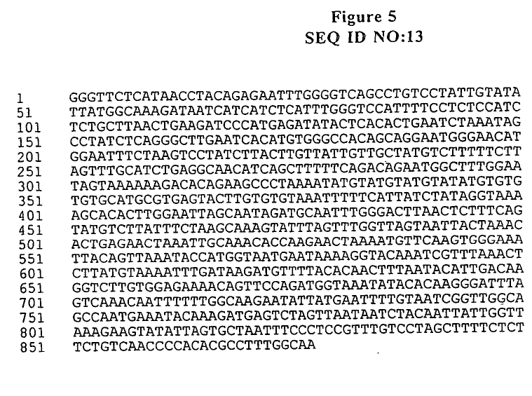

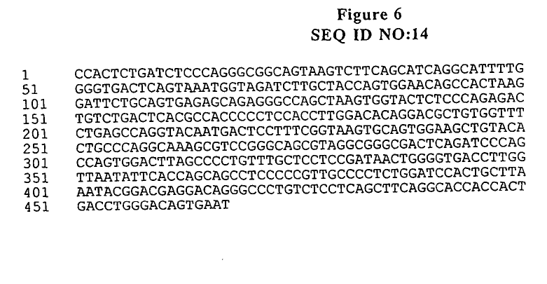

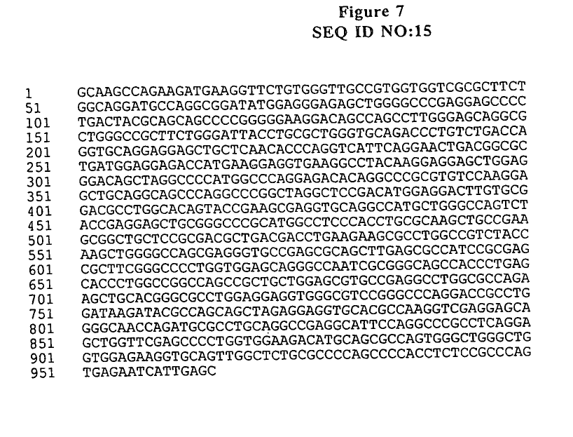

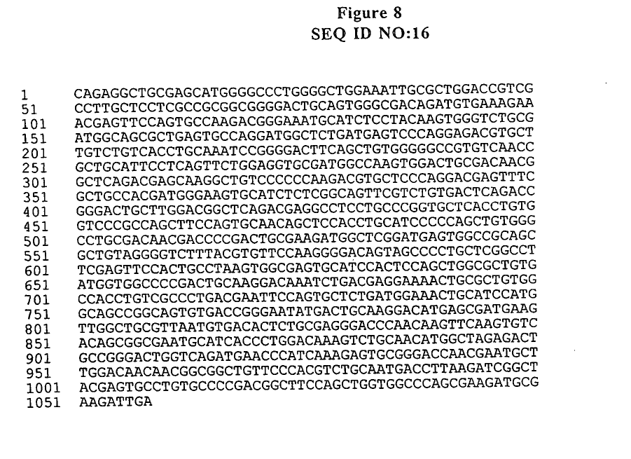

### DEFINITIONS

To facilitate understanding of the invention, a number of terms are defined below.

As used herein, the term “host cell” refers to any eukaryotic cell (e.g. mammalian cells, avian cells, amphibian cells, plant cells, fish cells, and insect cells), whether located in vitro or in vivo.

As used herein, the term “cell culture” refers to any in vitro culture of cells. Included within this term are continuous cell lines (e.g., with an immortal phenotype), primary cell cultures, finite cell lines (e.g., non-transformed cells), and any other cell population maintained in vitro, including oocytes and embryos.

As used herein, the term “vector” refers to any genetic element, such as a plasmid, phage, transposon, cosmid, chromosome, virus, virion, etc., which is capable of replication when associated with the proper control elements and which can transfer gene sequences between cells. Thus, the term includes cloning and expression vehicles, as well as viral vectors.

As used herein, the term “gene that regulates metabolism” refers to genes encoding proteins that catalyze the metabolism of a compound (e.g., lipids, sugars, proteins, etc.).

As used herein, the term “integrating vector” refers to a vector whose integration or insertion into a nucleic acid (e.g., a chromosome) is accomplished via an integrase. Examples of “integrating vectors” include, but are not limited to, retroviral vectors, transposons, and adeno associated virus vectors.

As used herein, the term “integrated” refers to a vector that is stably inserted into the genome (i.e., into a chromosome) of a host cell.

The term “nucleotide sequence of interest” refers to any nucleotide sequence (e.g., RNA or DNA), the manipulation of which may be deemed desirable for any reason (e.g., treat disease, confer improved qualities, etc.), by one of ordinary skill in the art. Such nucleotide sequences include, but are not limited to, coding sequences of structural genes (e.g., reporter genes, selection marker genes, oncogenes, drug resistance genes, growth factors, etc.), and non-coding regulatory sequences which do not encode an mRNA or protein product (e.g., promoter sequence, polyadenylation sequence, termination sequence, enhancer sequence, etc.).

As used herein, the term “protein of interest” refers to a protein encoded by a nucleic acid of interest.

As used herein, the term “exogenous gene” refers to a gene that is not naturally present in a host organism or cell, or is artificially introduced into a host organism or cell.

The term “gene” refers to a nucleic acid (e.g., DNA or RNA) sequence that comprises coding sequences necessary for the production of a polypeptide or precursor (e.g. proinsulin). The polypeptide can be encoded by a full length coding sequence or by any portion of the coding sequence so long as the desired activity or functional properties (e.g., enzymatic activity, ligand binding, signal transduction, etc.) of the full-length or fragment are retained. The term also encompasses the coding region of a structural gene and includes sequences located adjacent to the coding region on both the 5′ and 3′ ends for a distance of about 1 kb or more on either end such that the gene corresponds to the length of the full-length mRNA. The sequences that are located 5′ of the coding region and which are present on the mRNA are referred to as 5′ untranslated sequences. The sequences that are located 3′ or downstream of the coding region and which are present on the mRNA are referred to as 3′ untranslated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene which are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “gene expression” refers to the process of converting genetic information encoded in a gene into RNA (e.g., mRNA, rRNA, tRNA, or snRNA) through “transcription” of the gene (i.e., via the enzymatic action of an RNA polymerase), and for protein encoding genes, into protein through “translation” of mRNA. Gene expression can be regulated at many stages in the process. “Up-regulation” or “activation” refers to regulation that increases the production of gene expression products (ie., RNA or protein), while “down-regulation” or “repression” refers to regulation that decrease production. Molecules (e.g., transcription factors) that are involved in up-regulation or down-regulation are often called “activators” and “repressors,” respectively.

Where “amino acid sequence” is recited herein to refer to an amino acid sequence of a naturally occurring protein molecule, “amino acid sequence” and like terms, such as “polypeptide” or “protein” are not meant to limit the amino acid sequence to the complete, native amino acid sequence associated with the recited protein molecule.

As used herein, the terms “nucleic acid molecule encoding,” “DNA sequence encoding,” “DNA encoding,” “RNA sequence encoding,” and “RNA encoding” refer to the order or sequence of deoxyribonucleotides or ribonucleotides along a strand of deoxyribonucleic acid or ribonucleic acid. The order of these deoxyribonucleotides or ribonucleotides determines the order of amino acids along the polypeptide (protein) chain. The DNA or RNA sequence thus codes for the amino acid sequence.

As used herein, the term “variant,” when used in reference to a protein, refers to proteins encoded by partially homologous nucleic acids so that the amino acid sequence of the proteins varies. As used herein, the term “variant” encompasses proteins encoded by homologous genes having both conservative and nonconservative amino acid substitutions that do not result in a change in protein function, as well as proteins encoded by homologous genes having amino acid substitutions that cause decreased (e.g., null mutations) protein function or increased protein function.

As used herein, the terms “complementary” or “complementarity” are used in reference to polynucleotides (i.e., a sequence of nucleotides) related by the base-pairing rules. For example, for the sequence “A-G-T,” is complementary to the sequence “T-C-A.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

The terms “homology” and “percent identity” when used in relation to nucleic acids refers to a degree of complementarity. There may be partial homology (ie., partial identity) or complete homology (i.e., complete identity). A partially complementary sequence is one that at least partially inhibits a completely complementary sequence from hybridizing to a target nucleic acid sequence and is referred to using the functional term “substantially homologous.” The inhibition of hybridization of the completely complementary sequence to the target sequence may be examined using a hybridization assay (Southern or Northern blot, solution hybridization and the like) under conditions of low stringency. A substantially homologous sequence or probe (i.e., an oligonucleotide which is capable of hybridizing to another oligonucleotide of interest) will compete for and inhibit the binding (i.e., the hybridization) of a completely homologous sequence to a target sequence under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target which lacks even a partial degree of complementarity (e.g., less than about 30% identity); in the absence of non-specific binding the probe will not hybridize to the second non-complementary target.

The art knows well that numerous equivalent conditions may be employed to comprise low stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol) are considered and the hybridization solution may be varied to generate conditions of low stringency hybridization different from, but equivalent to, the above listed conditions. LI addition, the art knows conditions that promote hybridization under conditions of high stringency (e.g., increasing the temperature of the hybridization and/or wash steps, the use of formamide in the hybridization solution, etc.).

When used in reference to a double-stranded nucleic acid sequence such as a cDNA or genomic clone, the term “substantially homologous” refers to any probe that can hybridize to either or both strands of the double-stranded nucleic acid sequence under conditions of low stringency as described above.

When used in reference to a single-stranded nucleic acid sequence, the term “substantially homologous” refers to any probe that can hybridize (ie., it is the complement of) the single-stranded nucleic acid sequence under conditions of low stringency as described above.

As used herein, the term “hybridization” is used in reference to the pairing of complementary nucleic acids. Hybridization and the strength of hybridization (i.e., the strength of the association between the nucleic acids) is impacted by such factors as the degree of complementary between the nucleic acids, stringency of the conditions involved, the Tₘ of the formed hybrid, and the G:C ratio within the nucleic acids. A single molecule that contains pairing of complementary nucleic acids within its structure is said to be “self-hybridized.”

As used herein, the term “Tₘ” is used in reference to the “melting temperature” of a nucleic acid. The melting temperature is the temperature at which a population of double-stranded nucleic acid molecules becomes half dissociated into single strands. The equation for calculating the Tₘ of nucleic acids is well known in the art. As indicated by standard references, a simple estimate of the Tₘ value may be calculated by the equation: Tₘ=81.5+0.41(% G+C), when a nucleic acid is in aqueous solution at 1 M NaCl (See e.g., Anderson and Young, Quantitative Filter Hybridization, in Nucleic Acid Hybridization [1985]). Other references include more sophisticated computations that take structural as well as sequence characteristics into account for the calculation of Tₘ.

As used herein the term “stringency” is used in reference to the conditions of temperature, ionic strength, and the presence of other compounds such as organic solvents, under which nucleic acid hybridizations are conducted. With “high stringency” conditions, nucleic acid base pairing will occur only between nucleic acid fragments that have a high frequency of complementary base sequences. Thus, conditions of “weak” or “low” stringency are often required with nucleic acids that are derived from organisms that are genetically diverse, as the frequency of complementary sequences is usually less.

“High stringency conditions” when used in reference to nucleic acid hybridization comprise conditions equivalent to binding or hybridization at 42° C. in a solution consisting of 5× SSPE (43.8 g/l NaCl, 6.9 g/l NaH₂PO₄.H₂O and 1.85 g/l EDTA, pH adjusted to 7.4 with NaOH), 0.5% SDS, 5× Denhardt's reagent and 100 μg/ml denatured salmon sperm DNA followed by washing in a solution comprising 0.1× SSPE, 1.0% SDS at 42° C. when a probe of about 500 nucleotides in length is employed.

“Medium stringency conditions” when used in reference to nucleic acid hybridization comprise conditions equivalent to binding or hybridization at 42° C in a solution consisting of 5× SSPE (43.8 g/l NaCl, 6.9 g/l NaH₂PO₄.H₂O and 1.85 g/l EDTA, pH adjusted to 7.4 with NaOH), 0.5% SDS, 5× Denhardt's reagent and 100 μg/ml denatured salmon sperm DNA followed by washing in a solution comprising 1.0× SSPE, 1.0% SDS at 42° C. when a probe of about 500 nucleotides in length is employed.

“Low stringency conditions” comprise conditions equivalent to binding or hybridization at 42° C in a solution consisting of 5×SSPE (43.8 g/l NaCl, 6.9 g/l NaH₂PO₄.H₂O and 1.85 g/l EDTA, pH adjusted to 7.4 with NaOH), 0.1% SDS, 5× Denhardt's reagent [50× Denhardt's contains per 500 ml: 5 g Ficoll (Type 400, Pharamcia), 5 g BSA (Fraction V; Sigma)] and 100 μg/ml denatured salmon sperm DNA followed by washing in a solution comprising 5× SSPE, 0.1% SDS at 42° C. when a probe of about 500 nucleotides in length is employed.

A gene may produce multiple RNA species that are generated by differential splicing of the primary RNA transcript. cDNAs that are splice variants of the same gene will contain regions of sequence identity or complete homology (representing the presence of the same exon or portion of the same exon on both cDNAs) and regions of complete non-identity (for example, representing the presence of exon “A” on cDNA 1 wherein cDNA 2 contains exon “B” instead). Because the two cDNAs contain regions of sequence identity they will both hybridize to a probe derived from the entire gene or portions of the gene containing sequences found on both cDNAs; the two splice variants are therefore substantially homologous to such a probe and to each other.

The terms “in operable combination,” “in operable order,” and “operably linked” as used herein refer to the linkage of nucleic acid sequences in such a manner that a nucleic acid molecule capable of directing the transcription of a given gene and/or the synthesis of a desired protein molecule is produced. The term also refers to the linkage of amino acid sequences in such a manner so that a functional protein is produced.

As used herein, the term “selectable marker” refers to a gene that encodes an enzymatic activity that confers the ability to grow in medium lacking what would otherwise be an essential nutrient (e.g. the HIS3 gene in yeast cells); in addition, a selectable marker may confer resistance to an antibiotic or drug upon the cell in which the selectable marker is expressed. Selectable markers may be “dominant”; a dominant selectable marker encodes an enzymatic activity that can be detected in any eukaryotic cell line. Examples of dominant selectable markers include the bacterial aminoglycoside 3′ phosphotransferase gene (also referred to as the neo gene) that confers resistance to the drug G418 in mammalian cells, the bacterial hygromycin G phosphotransferase (hyg) gene that confers resistance to the antibiotic hygromycin and the bacterial xanthine-guanine phosphoribosyl transferase gene (also referred to as the gpt gene) that confers the ability to grow in the presence of mycophenolic acid. Other selectable markers are not dominant in that their use must be in conjunction with a cell line that lacks the relevant enzyme activity. Examples of non-dominant selectable markers include the thymidine kinase (tk) gene that is used in conjunction with tk⁻ cell lines, the CAD gene which is used in conjunction with CAD-deficient cells and the mammalian hypoxanthine-guanine phosphoribosyl transferase (hprt) gene which is used in conjunction with hprt⁻ cell lines. A review of the use of selectable markers in mammalian cell lines is provided in Sambrook, J. et al., Molecular Cloning: A Laboratory Manual, 2nd ed., Cold Spring Harbor Laboratory Press, New York (1989) pp.16.9-16.15.

As used herein, the term “regulatory element” refers to a genetic element which controls some aspect of the expression of nucleic acid sequences. For example, a promoter is a regulatory element that facilitates the initiation of transcription of an operably linked coding region. Other regulatory elements are splicing signals, polyadenylation signals, termination signals, RNA export elements, internal ribosome entry sites, etc. (defined infra).

Transcriptional control signals in eukaryotes comprise “promoter” and “enhancer” elements. Promoters and enhancers consist of short arrays of DNA sequences that interact specifically with cellular proteins involved in transcription (Maniatis et al., Science 236:1237 [1987]). Promoter and enhancer elements have been isolated from a variety of eukaryotic sources including genes in yeast, insect and mammalian cells, and viruses (analogous control elements, ie., promoters, are also found in prokaryotes). The selection of a particular promoter and enhancer depends on what cell type is to be used to express the protein of interest. Some eukaryotic promoters and enhancers have a broad host range while others are functional in a limited subset of cell types (for review see, Voss et al., Trends Biochem. Sci., 11:287 [1986]; and Maniatis et al., supra). For example, the SV40 early gene enhancer is very active in a wide variety of cell types from many mammalian species and has been widely used for the expression of proteins in mammalian cells (Dijkema et al., EMBO J. 4:761 [1985]). Two other examples of promoter/enhancer elements active in a broad range of mammalian cell types are those from the human elongation factor la gene (Uetsuki et al., J. Biol. Chem., 264:5791 [1989]; Kim et al., Gene 91:217 [1990]; and Mizushima and Nagata, Nuc. Acids. Res., 18:5322 [1990]) and the long terminal repeats of the Rous sarcoma virus (Gorman et al., Proc. Natl. Acad. Sci. USA 79:6777 [1982]) and the human cytomegalovirus (Boshart et al., Cell 41:521 [1985]).

As used herein, the term “promoter/enhancer” denotes a segment of DNA which contains sequences capable of providing both promoter and enhancer functions (i.e., the functions provided by a promoter element and an enhancer element, see above for a discussion of these functions). For example, the long terminal repeats of retrovimises contain both promoter and enhancer functions. The enhancer/promoter may be “endogenous” or “exogenous” or “heterologous.” An “endogenous” enhancer/promoter is one which is naturally linked with a given gene in the genome. An “exogenous” or “heterologous” enhancer/promoter is one which is placed in juxtaposition to a gene by means of genetic manipulation (i.e., molecular biological techniques such as cloning and recombination) such that transcription of that gene is directed by the linked enhancer/promoter.

Regulatory elements may be tissue specific or cell specific. The term “tissue specific” as it applies to a regulatory element refers to a regulatory element that is capable of directing selective expression of a nucleotide sequence of interest to a specific type of tissue (e.g., liver) in the relative absence of expression of the same nucleotide sequence of interest in a different type of tissue (e.g., lung).

Tissue specificity of a regulatory element may be evaluated by, for example, operably linking a reporter gene to a promoter sequence (which is not tissue-specific) and to the regulatory element to generate a reporter construct, introducing the reporter construct into the genome of an animal such that the reporter construct is integrated into every tissue of the resulting transgenic animal, and detecting the expression of the reporter gene (e.g., detecting mRNA, protein, or the activity of a protein encoded by the reporter gene) in different tissues of the transgenic animal. The detection of a greater level of expression of the reporter gene in one or more tissues relative to the level of expression of the reporter gene in other tissues shows that the regulatory element is “specific” for the tissues in which greater levels of expression are detected. Thus, the term “tissue-specific” (e.g., liver-specific) as used herein is a relative term that does not require absolute specificity of expression. In other words, the term “tissue-specific” does not require that one tissue have extremely high levels of expression and another tissue have no expression. It is sufficient that expression is greater in one tissue than another. By contrast, “strict” or “absolute” tissue-specific expression is meant to indicate expression in a single tissue type (e.g., liver) with no detectable expression in other tissues.

The term “cell type specific” as applied to a regulatory element refers to a regulatory element which is capable of directing selective expression of a nucleotide sequence of interest in a specific type of cell in the relative absence of expression of the same nucleotide sequence of interest in a different type of cell within the same tissue. The term “cell type specific” when applied to a regulatory element also means a regulatory element capable of promoting selective expression of a nucleotide sequence of interest in a region within a single tissue.

Cell type specificity of a regulatory element may be assessed using methods well known in the art (e.g., immunohistochemical staining and/or Northern blot analysis). Briefly, for immunohistochemical staining, tissue sections are embedded in paraffin, and paraffin sections are reacted with a primary antibody specific for the polypeptide product encoded by the nucleotide sequence of interest whose expression is regulated by the regulatory element. A labeled (e.g., peroxidase conjugated) secondary antibody specific for the primary antibody is allowed to bind to the sectioned tissue and specific binding detected (e.g., with avidin/biotin) by microscopy. Briefly, for Northern blot analysis, RNA is isolated from cells and electrophoresed on agarose gels to fractionate the RNA according to size followed by transfer of the RNA from the gel to a solid support (e.g., nitrocellulose or a nylon membrane). The immobilized RNA is then probed with a labeled oligo-deoxyribonucleotide probe or DNA probe to detect RNA species complementary to the probe used. Northern blots are a standard tool of molecular biologists.

The term “promoter,” “promoter element,” or “promoter sequence” as used herein, refers to a DNA sequence which when ligated to a nucleotide sequence of interest is capable of controlling the transcription of the nucleotide sequence of interest into mRNA. A promoter is typically, though not necessarily, located 5′ (i.e., upstream) of a nucleotide sequence of interest whose transcription into mRNA it controls, and provides a site for specific binding by RNA polymerase and other transcription factors for initiation of transcription.

Promoters may be constitutive or regulatable. The term “constitutive” when made in reference to a promoter means that the promoter is capable of directing transcription of an operably linked nucleic acid sequence in the absence of a stimulus (e.g. heat shock, chemicals, etc.). In contrast, a “regulatable” promoter is one which is capable of directing a level of transcription of an operably linked nucleic acid sequence in the presence of a stimulus (e.g., heat shock, chemicals, etc.) which is different from the level of transcription of the operably linked nucleic acid sequence in the absence of the stimulus.

The presence of “splicing signals” on an expression vector often results in higher levels of expression of the recombinant transcript. Splicing signals mediate the removal of introns from the primary RNA transcript and consist of a splice donor and acceptor site (Sambrook et al., Molecular Cloning: A Laboratory Manual, 2nd ed., Cold Spring Harbor Laboratory Press, New York [1989], pp. 16.7-16.8). A commonly used splice donor and acceptor site is the splice junction from the 16S RNA of SV40.

Efficient expression of recombinant DNA sequences in eukaryotic cells requires expression of signals directing the efficient termination and polyadenylation of the resulting transcript. Transcription termination signals are generally found downstream of the polyadenylation signal and are a few hundred nucleotides in length. The term “poly A site” or “poly A sequence” as used herein denotes a DNA sequence that directs both the termination and polyadenylation of the nascent RNA transcript. Efficient polyadenylation of the recombinant transcript is desirable as transcripts lacking a poly A tail are unstable and are rapidly degraded. The poly A signal utilized in an expression vector may be “heterologous” or “endogenous.” An endogenous poly A signal is one that is found naturally at the 3′ end of the coding region of a given gene in the genome. A heterologous poly A signal is one that is isolated from one gene and placed 3′ of another gene. A commonly used heterologous poly A signal is the SV40 poly A signal. The SV40 poly A signal is contained on a 237 bp BamHI/BclI restriction fragment and directs both termination and polyadenylation (Sambrook, supra, at 16.6-16.7).

Eukaryotic expression vectors may also contain “viral replicons ” or “viral origins of replication.” Viral replicons are viral DNA sequences that allow for the extrachromosomal replication of a vector in a host cell expressing the appropriate replication factors. Vectors that contain either the SV40 or polyoma virus origin of replication replicate to high “copy number” (up to 10⁴ copies/cell) in cells that express the appropriate viral T antigen. Vectors that contain the replicons from bovine papillomavirus or Epstein-Barr virus replicate extrachromosomally at “low copy number” (˜100 copies/cell). However, it is not intended that expression vectors be limited to any particular viral origin of replication.

As used herein, the term “long terminal repeat” of “LTR” refers to transcriptional control elements located in or isolated from the U3 region 5′ and 3′ of a retroviral genome. As is known in the art, long terminal repeats may be used as control elements in retroviral vectors, or isolated from the retroviral genome and used to control expression from other types of vectors.

As used herein, the term “secretion signal” refers to any DNA sequence which when operably linked to a recombinant DNA sequence encodes a signal peptide which is capable of causing the secretion of the recombinant polypeptide. In general, the signal peptides comprise a series of about 15 to 30 hydrophobic amino acid residues (See, e.g., Zwizinski et al., J. Biol. Chem. 255(16): 7973-77 [1980], Gray et al., Gene 39(2): 247-54 [1985], and Martial et al., Science 205: 602-607 [1979]). Such secretion signal sequences are preferably derived from genes encoding polypeptides secreted from the cell type targeted for tissue-specific expression (e.g., secreted milk proteins for expression in and secretion from mammary secretory cells). Secretory DNA sequences, however, are not limited to such sequences. Secretory DNA sequences from proteins secreted from many cell types and organisms may also be used (e.g., the secretion signals for t-PA, serum albumin, lactoferrin, and growth hormone, and secretion signals from microbial genes encoding secreted polypeptides such as from yeast, filamentous fungi, and bacteria).

As used herein, the terms “RNA export element” or “Pre-mRNA Processing Enhancer (PPE)” refer to 3′ and 5′ cis-acting post-transcriptional regulatory elements that enhance export of RNA from the nucleus. “PPE” elements include, but are not limited to Mertz sequences (described in U.S. Pat. Nos. 5,914,267 and 5,686,120, all of which are incorporated herein by reference) and woodchuck mRNA processing enhancer (WPRE; WO99/143 10, incorporated herein by reference).

As used herein, the term “polycistronic” refers to an mRNA encoding more than polypeptide chain (See, e.g., WO 93/03143, WO 88/05486, and European Pat. No. 117058, all of which is incorporated herein by reference). Likewise, the term “arranged in polycistronic sequence” refers to the arrangement of genes encoding two different polypeptide chains in a single mRNA.

As used herein, the term “internal ribosome entry site” or “IRES” refers to a sequence located between polycistronic genes that permits the production of the expression product originating from the second gene by internal initiation of the translation of the dicistronic mRNA. Examples of internal ribosome entry sites include, but are not limited to, those derived from foot and mouth disease virus (FDV), encephalomyocarditis virus, poliovirus and RDV (Scheper et al., Biochem. 76: 801-809 [1994]; Meyer et al., J. Virol. 69: 2819-2824 [1995]; Jang et al., 1988, J. Virol. 62: 2636-2643 [1998]; Haller et al., J. Virol. 66: 5075-5086 [1995]). Vectors incorporating IRES's may be assembled as is known in the art. For example, a retroviral vector containing a polycistronic sequence may contain the following elements in operable association: nucleotide polylinker, gene of interest, an internal ribosome entry site and a mammalian selectable marker or another gene of interest. The polycistronic cassette is situated within the retroviral vector between the 5′ LTR and the 3′ LTR at a position such that transcription from the 5′ LTR promoter transcribes the polycistronic message cassette. The transcription of the polycistronic message cassette may also be driven by an internal promoter (e.g., cytomegalovirus promoter) or an inducible promoter, which may be preferable depending on the use. The polycistronic message cassette can further comprise a cDNA or genomic DNA (gDNA) sequence operatively associated within the polylinker. Any mammalian selectable marker can be utilized as the polycistronic message cassette mammalian selectable marker. Such mammalian selectable markers are well known to those of skill in the art and can include, but are not limited to, kanamycin/G418, hygromycin B or mycophenolic acid resistance markers.

As used herein, the term “retrovirus” refers to a retroviral particle which is capable of entering a cell (i.e., the particle contains a membrane-associated protein such as an envelope protein or a viral G glycoprotein which can bind to the host cell surface and facilitate entry of the viral particle into the cytoplasm of the host cell) and integrating the retroviral genome (as a double-stranded provirus) into the genome of the host cell.

As used herein, the term “retroviral vector” refers to a retrovirus that has been modified to express a gene of interest. Retroviral vectors can be used to transfer genes efficiently into host cells by exploiting the viral infectious process. Foreign or heterologous genes cloned (i.e., inserted using molecular biological techniques) into the retroviral genome can be delivered efficiently to host cells which are susceptible to infection by the retrovirus. Through well known genetic manipulations, the replicative capacity of the retroviral genome can be destroyed. The resulting replication-defective vectors can be used to introduce new genetic material to a cell but they are unable to replicate. A helper virus or packaging cell line can be used to permit vector particle assembly and egress from the cell. Such retroviral vectors comprise a replication-deficient retroviral genome containing a nucleic acid sequence encoding at least one gene of interest (i.e., a polycistronic nucleic acid sequence can encode more than one gene of interest), a 5′ retroviral long terminal repeat (5′ LTR); and a 3′ retroviral long terminal repeat (3′ LTR).

The term “pseudotyped retroviral vector” refers to a retroviral vector containing a heterologous membrane protein. The term “membrane-associated protein” refers to a protein (e.g., a viral envelope glycoprotein or the G proteins of viruses in the Rhabdoviridae family such as VSV, Piry, Chandipura and Mokola) which are associated with the membrane surrounding a viral particle; these membrane-associated proteins mediate the entry of the viral particle into the host cell. The membrane associated protein may bind to specific cell surface protein receptors, as is the case for retroviral envelope proteins or the membrane-associated protein may interact with a phospholipid component of the plasma membrane of the host cell, as is the case for the G proteins derived from members of the Rhabdoviridae family.

The term “heterologous membrane-associated protein” refers to a membrane-associated protein which is derived from a virus which is not a member of the same viral class or family as that from which the nucleocapsid protein of the vector particle is derived. “Viral class or family” refers to the taxonomic rank of class or family, as assigned by the International Committee on Taxonomy of Viruses.

The term “Rhabdoviridae” refers to a family of enveloped RNA viruses that infect animals, including humans, and plants. The Rhabdoviridae family encompasses the genus Vesiculovirus which includes vesicular stomatitis virus (VSV), Cocal virus, Piry virus, Chandipura virus, and Spring viremia of carp virus (sequences encoding the Spring viremia of carp virus are available under GenBank accession number U18101). The G proteins of viruses in the Vesiculovirus genera are virally-encoded integral membrane proteins that form externally projecting homotrimeric spike glycoproteins complexes that are required for receptor binding and membrane fusion. The G proteins of viruses in the Vesiculovirus genera have a covalently bound palmititic acid (C₁₆) moiety. The amino acid sequences of the G proteins from the Vesiculoviruses are fairly well conserved. For example, the Piry virus G protein share about 38% identity and about 55% similarity with the VSV G proteins (several strains of VSV are known, e.g., Indiana, New Jersey, Orsay, San Juan, etc., and their G proteins are highly homologous). The Chandipura virus G protein and the VSV G proteins share about 37% identity and 52% similarity. Given the high degree of conservation (amino acid sequence) and the related functional characteristics (e.g., binding of the virus to the host cell and fusion of membranes, including syncytia formation) of the G proteins of the Vesiculoviruses, the G proteins from non-VSV Vesiculoviruses may be used in place of the VSV G protein for the pseudotyping of viral particles. The G proteins of the Lyssa viruses (another genera within the Rhabdoviridae family) also share a fair degree of conservation with the VSV G proteins and function in a similar manner (e.g., mediate fusion of membranes) and therefore may be used in place of the VSV G protein for the pseudotyping of viral particles. The Lyssa viruses include the Mokola virus and the Rabies viruses (several strains of Rabies virus are known and their G proteins have been cloned and sequenced). The Mokola virus G protein shares stretches of homology (particularly over the extracellular and transmembrane domains) with the VSV G proteins which show about 31% identity and 48% similarity with the VSV G proteins. Preferred G proteins share at least 25% identity, preferably at least 30% identity and most preferably at least 35% identity with the VSV G proteins. The VSV G protein from which New Jersey strain (the sequence of this G protein is provided in GenBank accession numbers M27165 and M21557) is employed as the reference VSV G protein.

As used herein, the term “lentivirus vector” refers to retroviral vectors derived from the Lentiviridae family (e.g., human immunodeficiency virus, simian immunodeficiency virus, equine infectious anemia virus, and caprine arthritis-encephalitis virus) that are capable of integrating into non-dividing cells (See, e.g., U.S. Pat. Nos. 5,994,136 and 6,013,516, both of which are incorporated herein by reference).

The term “pseudotyped lentivirus vector” refers to lentivirus vector containing a heterologous membrane protein (e.g., a viral envelope glycoprotein or the G proteins of viruses in the Rhabdoviridae family such as VSV, Piry, Chandipura and Mokola).

As used herein, the term “purified” refers to molecules, either nucleic or amino acid sequences, that are removed from their natural environment, isolated or separated. An “isolated nucleic acid sequence” is therefore a purified nucleic acid sequence. “Substantially purified” molecules are at least 60% free, preferably at least 75% free, and more preferably at least 90% free from other components with which they are naturally associated.

As used herein, the term “increased resistance to fatty liver disease” refers to a decreased risk of developing “fatty liver disease.” Symptoms of fatty liver disease include, but are not limited to increased susceptibility to opportunistic infectious diseases of the udder and uterus, decreased appetite, impaired urea metabolism, increased susceptibility to ketosis, reduced fertility, increased risk of displaced abomasum, metritis, retained placenta, and increased risk of non responding hypocalcinemia, or milk fever. “Increased resistance to fatty liver disease” results from alterations in lipid metabolism, including but not limited to “increased lipid motility” and “increased level of apoB secretion.” As used herein, the term “increased lipid mobility” refers to a higher rate of hepatic triglyceride export and, therefore, a faster rate of triglyceride accumulation in blood.

As used herein “increased level of apoB secretion” refers to an increase in the ability of cells (e.g., liver cells) to secrete triglycerides (e.g., apolipoprotein B [apoB]) on lipoprotein (e.g., low density lipoprotein or very low density lipoprotein) particles.

### DETAILED DESCRIPTION OF THE INVENTION

In some embodiments, the present inventions provides methods for generating bovines resistant to fatty liver disease. In other embodiments, the present invention provides transgenic bovines resistant to fatty liver disease. In some embodimnents, the bovines are generated by retroviral vector infection of oocytes or zygotes. The retroviral vectors further comprise a liver-specific promoter and a gene coding for a protein involved in lipoprotein metabolism, including but not limited to truncated soluble LDL receptor and ApoE.

The present invention thus provides an efficient, cost effective method of generating transgenic bovines resistant to fatty liver disease. The fatty liver disease resistant bovines of the present invention provide a large cost-savings to the dairy industry, both in cost of generating the transgenic bovines and in lost revenues due to disease.

### I. Fatty Liver Disease in Cows

In some embodiments, the present invention provides methods and compositions for generating transgenic bovines resistant to fatty liver disease (e.g., bovines with increased lipid mobility). In general, fatty liver disease is caused by an imbalance between the liver's ability to produce triglycerides and its ability to secrete triglycerides on very low density lipoprotein (VLDL) particles. However, the present invention is not limited to any particular mechanism. In fact, an understanding of the mechanism is not necessary to practice the present invention. Nonetheless, it is believed that dairy cows are vulnerable to fatty liver disease, at least in part because they have a very limited capacity to secrete VLDL. In some embodiments, the present invention provides bovines comprising a transgene that overcomes this limitation. Thus, the transgenic bovines are less susceptible to fatty liver disease (e.g., they have increased lipid mobility and increase apoB secretion). In some embodiments, the present invention provides transgenic bovines that are less susceptible to ketosis. Thus, the present invention provides remedies for at least two of the most important metabolic disorders affecting dairy cows.

### A. LDL Receptors as a Regulator of VLDL Secretion

In some embodiments, the present invention provides bovines comprising transgenes expressing a soluble low density lipoprotein (LDL) receptor. Apolipoprotein B (apoB) is the major protein component of very low density lipoprotein (VLDL) and LDL. The LDL receptor mediates the clearance of LDL from the circulation by binding to apoB.

The present invention is not limited to a particular mechanism. In fact, an understanding of the mechanism is not necessary to practice the present invention. A soluble (i.e. not membrane-bound) fragment of the receptor dramatically increases the secretion of apoB in a model tissue culture system for recombinant protein production (Dirlam et al., Protein Exp. and Pur., 8:489 [1996]). Thus, it is contemplated that a soluble LDL receptor functions in a similar manner in mammalian (e.g., bovine) liver. A transgene expressing soluble LDL provides a means to increase the capacity of a cow to secrete VLDL particles and thereby, reduce or abolish its susceptibility to fatty liver disease.

Humans with LDL receptor deficiency (familial hypercholesterolemia (FM patients) are known to overproduce VLDL. The present invention is not limited to a particular mechanism. In fact, an understanding of the mechanism is not necessary to practice the present invention. Mechanistic studies were performed by isolating hepatocytes from wild type and from LDL receptor-deficient (LDLR−/−) mice. The latter mice were created by disrupting the LDL receptor gene to generate a truncated, dysfunctional LDL receptor. While the rate of apoB secretion was 3-fold higher in the LDLR−/− hepatocytes than the wild type cells, the rate of apoB synthesis and the mRNA abundance was the same in both cell types. Pulse-chase experiments showed that this large difference in apoB secretion was due to a much higher rate of post-translational apoB degradation in the wild type cells.

As a proof of principle, the LDL receptor was “added back” by infecting the hepatocytes with an adenovirus harboring an LDL receptor cDNA. Because transcription of this gene was driven by a very active promoter (the cytomegalovirus (CMV) promoter), much higher levels of LDL receptor were achieved than is normally seen in wild type cells. In cells from both wild type and LDLR−/− mice, apoB secretion was virtually abolished.

The present invention is not limited to a particular mechanism. In fact, an understanding of the mechanism is not necessary to practice the present invention. Nonetheless, it is contemplated that the LDL receptor binds to apoB within the secretory pathway and targets it for degradation. This model is supported by studies showing that antibodies to the LDL receptor immunoprecipitate a complex of newly-synthesized apoB and the LDL receptor. This complex can be detected within 5 minutes of labeling cells with radioactive amino acids (e.g., in 5 minutes, apoB polypeptide chains are end-label initiated, whereas a full chain requires about 15 minutes for translation).

In addition, the ability of the LDL receptor to target apoB for degradation depends upon the receptor being bound to a membrane. The ligand binding domain of the LDL receptor (“soluble LDL receptor”) was expressed in baculovirus-infected insect cells. The cells expressed and secreted the protein as a soluble product that retained its ability to bind to apoB. This protein was expressed with two different constructs derived from apoB. Namely, the N-terminal 17% of apoB (“apoB17′) and a fusion protein consisting of apoB17 and the receptor binding domain of apoB (“B17(B69-79)′). Thus, the expression product was a receptor ligand or a portion of the protein not able to bind to the receptor. The soluble LDL receptor increased the secretion of the fusion protein 10-fold, while having no effect on the observed secretion of apoB17.

Accordingly, it is believed that a soluble receptor can greatly increase apoB secretion while a membrane-bound receptor decreases apoB secretion by targeting it for degradation.

### B. The Role of apoE in VLDL Secretion

In some embodiments, the present invention provides bovines comprising transgenes expressing apolipoprotein E. Apolipoprotein E (apoE) is found on VLDL particles and is also a ligand for the LDL receptor. Deletion of the gene encoding apoE results in impaired VLDL secretion in mice. Conversely, overexpression of the apoE gene promotes more VLDL secretion.

Apolipoprotein E (apoE) is a 35 kDa apolipoprotein found in VLDL, chylomicrons, and sometimes in HDL. Like apoB, it is a ligand for the LDL receptor. Chylomicron remnant particles depend upon apoE for their clearance from the circulation owing to the fact that the apoB found in chylomicrons, apoB48, is a poor ligand for the LDL receptor (Welty et al., Arherioscler. Thromb. Vasc. Biol., 17:881 [1997]; Ishibashi et al., P.N.A.S., 91:4431 1994]). In the absence of apoE, is massive accumulation of remnant lipoproteins in the circulation (Piedrahita et al., P.N.A.S., 89:4471 [1992]). However, there is also a defect in VLDL secretion (Kulpers et al., Circulation, 94S:I-159 [1996]). Conversely, overexpression of apoE leads to a higher level of VLDL secretion (Huang et al., J. Biol. Chem., 273:26388 [1998]).

The present invention is not limited to a particular mechanism. In fact, an understanding of the mechanism is not necessary to practice the present invention. Nonetheless, it is contemplated that apoE and apoB present in the secretory pathway of hepatocytes compete for binding to the LDL receptor. ApoE is a better ligand and therefore displaces apoB, leading to less retention and more secretion of apoB. Preliminary studies in hepatocytes from apoE −/− transgenic mice support this model. Based on this model, it is contemplated that high-level expression of apoE will lead to a high level of apoB secretion.

Cows express apoE, but they do so at a very low level (Yau et al., J. Mol. Evol., 32:469 [1991]; Brzozowska et al., Mammalian Genome, 4:53 [1993]; Brantmeier et al., Lipids, 23: [1988]). The present invention is not limited to a particular mechanism. In fact, an understanding of the mechanism is not necessary to practice the present invention. Nonetheless, it is contemplated that the limited capacity of cows to secrete VLDL is a consequence of their low level of apoE expression. It is also contemplated that increased expression of apoE will alleviate this deficit and restore a cow's VLDL secretory capacity to that of mammals with higher level of apoE expression. Indeed, it is contemplated that by overcoming this deficiency, treated animals will be less vulnerable to the development of fatty liver disease.

### II. Methods for Generation of Transgenic Bovines

In some embodiments, the present invention provides improved methods of generating transgenic bovines (e.g., transgenic bovines resistant to fatty liver disease). Transgenic animals were first reported in the early 1980's with the ground-breaking work of Brinster and Palmiter (Brinster et al., PNAS, 85:846 [1985]). The key constraint in utilization of transgenic traits in animal agriculture in a manner similar to that which has revolutionized crop agriculture is the generation of transgenic animals. Traditional methods of producing transgenic animals involve pronuclear microinjection and more recently nuclear transfer (See e.g., U.S. Pat. Nos. 5,496,720; 4,994,384; 5,633,076; 4,873,191; and PCT publications WO 97/07669; WO 97/07668; WO 95/17500; all of which are herein incorporated by reference). It is contemplated that in some embodiments, these methods may be used to generate the transgenic bovines of the present invention. However, in preferred embodiments, transduction with retroviral vectors (e.g., those described below) is used to generate the transgenic bovines.

### III. Vectors for Generation of Transgenic Bovines

In some embodiments, the present invention provides vectors (e.g., retroviral vectors) for the generation of transgenic bovines resistant to fatty liver disease. In preferred embodiments, the vectors comprise a gene that when expressed, confers fatty liver disease resistance (e.g., apoE or truncated soluble LDL receptor). In some preferred embodiments, the vectors further comprise tissue-specific promoters (e.g., for expression of proteins in the liver).

### A. Retroviral Vectors

In some embodiments, the present invention provides retroviral vectors for the generation of transgenic bovines (e.g., fatty liver disease resistant bovines). In preferred embodiments, the present invention provides replication defective retroviruses as a means of gene introduction.

Retroviral infection, in which the genetic information is transferred as an RNA molecule, was the earliest method used for gene transfer into embryos (Jaenisch et al., PNAS, 73:1260 [1976]). Repeated attempts over a number of years showed that the lack of control of gene dose and timing using replication competent retroviruses resulted in nearly all the animals born being genetic mosaics, with multiple and different gene insertion locations in different tissues (Jaenisch, Cell, 19:181 [1980]). The retroviral utilized in the present invention overcome many of the problems of vectors in use previously.

Retroviruses (family Retroviridae) are divided into three groups: the spumaviruses (e.g., human foamy virus); the lentiviruses (e.g., human immunodeficiency virus and sheep visna virus) and the oncoviruses (e.g., MLV, Rous sarcoma virus). Retroviruses are enveloped (i.e., surrounded by a host cell-derived lipid bilayer membrane) single-stranded RNA viruses which infect animal cells. When a retrovirus infects a cell, its RNA genome is converted into a double-stranded linear DNA form (i.e., it is reverse transcribed). The DNA form of the virus is then integrated into the host cell genome as a provirus. The provirus serves as a template for the production of additional viral genomes and viral mRNAs. Mature viral particles containing two copies of genomic RNA bud from the surface of the infected cell. The viral particle comprises the genomic RNA, reverse transcriptase and other pol gene products inside the viral capsid (which contains the viral gag gene products) which is surrounded by a lipid bilayer membrane derived from the host cell containing the viral envelope glycoproteins (also referred to as membrane-associated proteins).

The organization of the genomes of numerous retroviruses is well known to the art and this has allowed the adaptation of the retroviral genome to produce retroviral vectors. The production of a recombinant retroviral vector carrying a gene of interest is typically achieved in two stages.

First, the gene of interest is inserted into a retroviral vector which contains the sequences necessary for the efficient expression of the gene of interest (including promoter and/or enhancer elements which may be provided by the viral long terminal repeats (LTRs) or by an internal promoter/enhancer and relevant splicing signals), sequences required for the efficient packaging of the viral RNA into infectious virions (e.g. the packaging signal (Psi), the tRNA primer binding site (−PBS), the 3′ regulatory sequences required for reverse transcription (+PBS)) and the viral LTRs. The LTRs contain sequences required for the association of viral genomic RNA, reverse transcriptase and integrase functions, and sequences involved in directing the expression of the genomic RNA to be packaged in viral particles. For safety reasons, many recombinant retroviral vectors lack functional copies of the genes which are essential for viral replication (these essential genes are either deleted or disabled); therefore, the resulting virus is said to be replication defective.

Second, following the construction of the recombinant vector, the vector DNA is introduced into a packaging cell line. Packaging cell lines provide proteins required in trans for the packaging of the viral genomic RNA into viral particles having the desired host range (i.e., the viral-encoded gag, pol and env proteins). The host range is controlled, in part, by the type of envelope gene product expressed on the surface of the viral particle. Packaging cell lines may express ecotrophic, amphotropic or xenotropic envelope gene products. Alternatively, the packaging cell line may lack sequences encoding a viral envelope (env) protein. In this case the packaging cell line packages the viral genome into particles which lack a membrane-associated protein (e.g., an env protein). In order to produce viral particles containing a membrane associated protein which will permit entry of the virus into a cell, the packaging cell line containing the retroviral sequences is transfected with sequences encoding a membrane-associated protein (e.g., the G protein of vesicular stomatitis virus (VSV)). The transfected packaging cell then produces viral particles which contain the membrane-associated protein expressed by the transfected packaging cell line; these viral particles which contain viral genomic RNA derived from one virus encapsidated by the envelope proteins of another virus are said to be “pseudotyped” virus particles.

In some embodiments, the retroviral vectors of the present invention are further modified to include additional regulatory sequences. As described above, the retroviral vectors of the present invention include the following elements in operable association: a) a 5′ LTR; b) a packaging signal; c) a 3′ LTR and d) a nucleic acid encoding a protein of interest located between the 5′ and 3′ LTRs. In some embodiments of the present invention, the nucleic acid of interest may be arranged in opposite orientation to the 5′ LTR when transcription from an internal promoter is desired. In some embodiments, the retroviral vectors comprise one of several suitable internal promoters.

In other embodiments of the present invention, where secretion of the protein of interest is desired, the vectors are modified by including a signal peptide sequence in operable association with the protein of interest. The sequences of several suitable signal peptides are known to those in the art, including, but not limited to, those derived from tissue plasminogen activator, human growth hormone, lactoferrin, alpha-casein, and alpha-lactalbumin.

In other embodiments of the present invention, the vectors are modified by incorporating an RNA export element (See, e.g., U.S. Pat. Nos. 5,914,267 and 5,686,120 and WO99/14310, all of which are incorporated herein by reference) either 3′ or 5′ to the nucleic acid sequence encoding the protein of interest. It is contemplated that the use of RNA export elements allows high levels of expression of the protein of interest without incorporating splice signals or introns in the nucleic acid sequence encoding the protein of interest.

In still other embodiments, the vector further comprises at least one internal ribosome entry site (IRES) sequence. The sequences of several suitable IRES's are known to those in the art, including, but not limited to, those derived from foot and mouth disease virus (FMDV), encephalomyocarditis virus, and poliovirus. The IRES sequence can be interposed between two transcriptional units (e.g., nucleic acids encoding different proteins of interest or subunits of a multisubunit protein such as an antibody) to form a polycistronic sequence so that the two transcriptional units are transcribed from the same promoter.

In some embodiments, the retroviral vectors utilized in the present invention further comprise a selectable marker allowing selection of transformed cells. A number of selectable markers find use in the present invention, including, but not limited to the bacterial aminoglycoside 3′ phosphotransferase gene (also referred to as the neo gene) that confers resistance to the drug G418 in mammalian cells; the bacterial hygromycin G phosphotransferase (hyg) gene that confers resistance to the antibiotic hygromycin; and the bacterial xanthine-guanine phosphoribosyl transferase gene (also referred to as the gpt gene) that confers the ability to grow in the presence of mycophenolic acid. In some embodiments, the selectable marker gene is provided as part of polycistronic sequence that also encodes the protein of interest.

In still other embodiments the retroviral vectors utilized in the present invention comprise recombination elements recognized by a recombination system (e.g., the cre/loxP or flp recombinase systems, See, e.g., Hoess et al., Nucleic Acids Res. 14:2287-2300 [1986], O'Gorman et al., Science 251:1351-55 [1991], van Deursen et al., Proc. Natl. Acad. Sci. USA 92:7376-80 [1995], and U.S. Pat. No. 6,025,192, herein incorporated by reference). In these embodiments, after integration of the vectors into the genome of the host cell, the host cell is transiently transfected (e.g., by electroporation, lipofection, or microinjection) with either a recombinase enzyme (e.g., Cre recombinase) or a nucleic acid sequence encoding the recombinase enzyme and one or more nucleic acid sequences encoding a protein of interest flanked by sequences recognized by the recombination enzyme so that the nucleic acid sequence is inserted into the integrated vector.

The most commonly used recombinant retroviral vectors are derived from the amphotropic Moloney murine leukemia virus (MOMLV) (See e.g., Miller and Baltimore Mol. Cell. Biol. 6:2895 [1986]). The MoMLV system has several advantages: 1) this retrovirus can infect many different cell types, 2) established packaging cell lines are available for the production of recombinant MoMLV viral particles, and 3) the transferred genes are permanently integrated into the target cell chromosome. The established MoMLV vector systems comprise a DNA vector containing a small portion of the retroviral sequence (e.g., the viral long terminal repeat or “LTR” and the packaging or “psi” signal) and a packaging cell line. The gene to be transferred is inserted into the DNA vector. The viral sequences present on the DNA vector provide the signals necessary for the insertion or packaging of the vector RNA into the viral particle and for the expression of the inserted gene. The packaging cell line provides the proteins required for particle assembly (See e.g., Markowitz et al., J. Virol. 62:1120 [1988]).

### B. Promoters

In some preferred embodiments, the present invention provides retroviral vectors comprising promoters for the expression of proteins in the bovine liver. In preferred embodiments, these genetic elements are liver-specific, due to the presence of a liver-specific regulatory element that allows expression of the transgene in only the liver and in no other tissues.

In some embodiments, the bovine albumin promoter is utilized. In other embodiments, bovine α-1-antitrypsin genetic regulatory elements are utilized to drive expression of transgenes in the liver. Both of these elements have been shown to function normally in a replication defective retroviral system (Hafenrichter et al., Blood, 84:10:3394 [1994]). The regulatory elements for these two genes have also been used to control the expression of a number of transgenes in the livers of transgenic mice (Gay et al, Endocrinology, 138:2937 [1997]; Kawamura et al., Hepatology, 25:1014 [1997]; Yull et al., Transgenic Research, 4:70 [1995]). Transgenic protein production induced by these promoters are similar to levels of Apo E and truncated LDL receptor needed to reduce the “fatty liver” phenotype in mice.

### C. Genes of Interest

In some embodiments of the present invention, the vectors further provide a gene of interest. In preferred embodiments, expression of the transgene results in transgenic bovines resistant to fatty liver disease. In some embodiments, the gene of interest comprises a bovine truncated soluble LDL receptor. In other embodiments, the gene of interest comprises a bovine ApoE. Methods for inserting a gene of interest into the vectors of the present invention are well known in the art.

### IV. Production of Transgenic Bovines

In some embodiments, the present invention provides methods for the production of transgenic bovines (e.g., fatty liver disease resistant bovines). In some preferred embodiments, transgenic bovines are generated using the retroviral vectors, genes, and promoters described above.

Most retroviruses can only infect dividing cells because of a critical need for nuclear membrane breakdown to allow the pre-integration complex to contact the chromosomal DNA. The present invention is not limited to a particular mechanism. Indeed, an understanding of the mechanism is not necessary to practice the present invention. Nonetheless, it is contemplated that the nuclear membrane breakdown which occurs in the oocyte, during metaphase II (MII) of the second meiosis, provides a window in which integration readily occurs. High virus titer is also necessary in order to permit the vector to be concentrated into a small enough volume for oocyte injection.

On a per copy basis, retroviral vectors integrated by their normal biological integration system exhibit significantly higher levels of expression than produced using other means of genetic transformation (Schubeler et al., Biochemistry, 35:11160 [1996]). In addition, the methods of the present invention permit single copy gene insertions. Such insertions may occur at several independent sites in the genome and are transmitted in a standard Mendelian pattern upon subsequent breeding.

Unlike earlier attempts to utilize retroviruses as gene delivery vehicles in transgenesis, the methods of the present invention utilize replication defective “pseudoviruses” which are incapable of replication. Three critical components of a retrovirus are removed to prevent further replication of the vector. The gag and pol genes needed for viral replication and packaging are supplied by the packaging cell line used to grow the vector. Envelope protein needed for infectivity is supplied by transient transfection of the packaging cell line with envelope protein from the vesicular stomatitis virus to provide a one-time batch of infectious vector. Thus, the actual vector delivered to the oocyte is capable only of a single integration event. The integrated gene is transcribed like other indigenous cell genes, and the proteins it codes for are expressed, but no further viral replication can occur.

There is a prevailing belief that it is important to be able to put in large pieces of genomic material into transgenic animals. However, by utilizing viral promoter elements in conjunction with precisely engineered tissue specific promoter elements, the need to use large genetic constructs can be overcome. Naturally, genes occupy large stretches of chromatin. However, 90% of the chromosomal region occupied by the gene is non-coding and is occupied by introns that get spliced out and never leave the nucleus. In some genes there are distant “locus control regions” which are important in controlling the gene expression. However, in many cases these are ill-defined. The average protein chain is about 35 kdaltons. Perhaps less than 1% of all genes have coding sequences >100,000 kdaltons (˜3 kb coding region). The retroviral constructs of the present invention can accommodate inserts up to 8 kb, sufficient for the vast majority of proteins of interest.

The methods of the present invention overcome three major problems of the more traditional forms of transgenic animal production currently in use, pronuclear microinjection and nuclear transfer. Firstly, the efficiency of transgenic live births achieved in early trials approached 100% of cattle born. Secondly, because genes insert as single copies, there is less risk of genetic instability upon subsequent cell replication, which tends to splice out tandem repeats of genes typical of DNA injection technologies. Thirdly, because transgenes are inserted prior to fertilization, there is no risk of production of mosaics.

Gene introduction by injection into the perivitelline space of the bovine oocyte is considerably simpler than the more precise micromanipulation needed for pronuclear injection. Because the technique is so efficient, it reduces the need to maintain a large numbers of recipient cattle. Costs of transgenic animal production are thus greatly reduced. Also reduced is the risk of introducing disease into the founder herd, inherent in any system that sources a large number of cattle or oocytes from the general population.

In one illustrative example of the present invention, a vector constructed to comprise two MoMLV LTR sequences flanking the hepatitis surface antigen protein gene and a neomycin phosphotransferase expressed from Rous sarcoma virus (RSV) promoter was injected into the perivitelline space of bovine oocytes in metaphase II arrest. Of 836 oocytes injected, 174 developed to the blastocyst stage. Of these, 10 were selected for transfer in pairs to recipient bovines, resulting in 10 pregnancies and 4 live births. Of the 4 calves born, 3 were transgenic (Chan et al., PNAS, 1998). An additional male was derived by early zygote transduction of the same vector and also is a germline transmitter of the transgene. PCR and Southern blot hybridization test on tissues from multiple embryonic lines in each calf demonstrated identical patterns of insertion and transgenesis. Second generation offspring of the zygote derived male are transgenic.

Six healthy liveborn transgenic cattle (of seven calves born from five pregnancies), including two second generation calves have been generated using the methods of the present invention. All the cattle born are healthy and have developed normally. The two females now of lactational age are expressing the transgene protein (hepatitis B surface antigen) in their milk.

In some embodiments, the present invention provides methods and compositions for generating transgenic bovines resistant to fatty liver disease. Protocols for cloning the promoters and genes of interest are provided in illustrative Examples 1-4 below (See also, U.S. Pat. No. 6,080,912 and PCT publication WO 00/30437; each of which are incorporated herein by reference). Methods for generating and packaging the vectors of the present invention are described in illustrative Examples 5-6 below. In preferred embodiments, the vectors are evaluated for their ability to effect apoB secretion and triglyceride metabolism in hepatocytes. The expression of the proteins of interest (e.g., apoE or truncated soluble LDL receptor) is assayed in 293 cells. Illustrative Example 7, below, describes such assays. Following the evaluation of retroviral vectors expressing the gene of interest in cell culture, transgenic bovines are generated. Example 8 illustrates methods for embryo production, transfer, and gestation of transgenic bovines. PCR is used to identify transgenic offspring. Transgenic calves and control (non-transgenic) calves are evaluated for symptoms of fatty liver disease. Illustrative Example 8 describes methods for evaluating resistance to fatty liver disease. In preferred embodiments, transgenic bovines expressing a gene of interest exhibit increased resistance to fatty liver disease. In some embodiments, elite seedstock animals exhibiting resistance to fatty liver disease are generated.

The methods of the present invention make it possible to generate groups of transgenic embryos in each embryo culture dish. Thus it is possible to create multiple unique founder animals, to compare expression phenotypes, and then to apply traditional animal selection methods to expand the best line. From the perspective of transgenic animal production, this means it is possible to create production herds from the very best founder animals, rather than being forced to work with a single rare founder. When viewed from the perspective of enhanced livestock genetics, the simplicity and high efficiency of the present invention make it possible to add valuable genetic traits to oocytes harvested in relatively small numbers from donors of elite, highly selected genetic stock.

### Experimental

The following examples serve to illustrate certain preferred embodiments and aspects of the present invention and are not to be construed as limiting the scope thereof In the experimental disclosure which follows, the following abbreviations apply: M (molar); mM (millimolar); μM (micromolar); nM (nanomolar); mol (moles); mmol (millimoles); μmol (micromoles); nmol (nanomoles); gm (grams); mg (milligrams); μg (micrograms); pg (picograms); L (liters); ml (milliliters); μl (microliters); cm (centimeters); mm (millimeters); μm (micrometers); nm (nanometers); ° C. (degrees Centigrade); AMP (adenosine 5′-monophosphate); BSA (bovine serum albumin); cDNA (copy or complimentary DNA); CS (calf serum); DNA (deoxyribonucleic acid); ssDNA (single stranded DNA); dsDNA (double stranded DNA); DNTP (deoxyribonucleotide triphosphate); LH (luteinizing hormone); NIH (National Institutes of Health, Besthesda, Md.); RNA (ribonucleic acid); PBS (phosphate buffered saline); g (gravity); OD (optical density); HEPES (N-[2-Hydroxyethyl]piperazine-N-[2-ethanesulfonic acid]); HBS (HEPES buffered saline); PBS (phosphate buffered saline); SDS (sodium dodecylsulfate); Tris-HCl (tris[Hydroxymethyl]aminomethane-hydrochloride); Klenow (DNA polymerase I large (Klenow) fragment); rpm (revolutions per minute); EGTA (ethylene glycol-bis(β-aminoethyl ether) N, N, N′, N′-tetraacetic acid); EDTA (ethylenediaminetetraacetic acid); bla (β-lactamase or ampicillin-resistance gene); ORI (plasmid origin of replication); lacI (lac repressor); X-gal (5-bromo4-chloro-3-indolyl-β-D-galactoside); ATCC (American Type Culture Collection, Rockville, Md.); GIBCO/BRL (GIBCO/BRL, Grand Island, N.Y.); Perkin-Elmer (Perkin-Elmer, Norwalk, Conn.); and Sigma (Sigma Chemical Company, St. Louis, Mo.).

### EXAMPLE 1

### Isolation of Bovine ApoE Gene

First, mRNA is isolated from bovine liver (PolyATtract mRNA isolation system, Promega, Madison, Wis.). The mRNA is then reverse transcribed using the Access RT-PCR System, Promega, Madison, Wis. The cDNA is then amplified using polymerase chain reaction and primers (5′ GCGGTTGGCCTAGGGCAAGCCAGAAGATGAAGGTTCTGT 3′; SEQ ID NO: 1 and 5′ GGCGATGCGTCGACGCTCAATGATTCTCACTGGGCGGAGA 3′; SEQ ID NO: 2). PCR amplification is used to isolate the entire ApoE gene with an AvrII restriction site on the 5′ end of the gene and a Sal I restriction site on the 3′ end of the gene. This allows easy cloning into retroviral backbones.

### EXAMPLE 2

### Isolation of Bovine Truncated LDL Receptor

First, mRNA is isolated from the bovine adrenal gland (PolyATtract mRNA isolation system, Promega, Madison, Wis.). The mRNA is then reverse transcribed using the Access RT-PCR System, Promega, Madison, Wis. The cDNA in then amplified using polymerase chain reaction and primers: (5° CGGGACACTCCTAGGCAGAGGCTGCGAGCATGGGGCCCTG 3′, SEQ ID NO: 3 and 5° CTGTCACTCGTCGACTCAATCTTCGCATCTTCGCTGGGCCA 3′; SEQ ID NO:4). This PCR amplification creates a truncated LDL receptor gene with an AvrII restriction site on the 5′ end of the gene and a Sal I restriction site on the 3′ end of the gene, allowing easy cloning into retroviral backbones. The truncated bovine LDL receptor gene codes for a 354 amino acid pre-protein and after signal peptide cleavage the mature protein is 333 amino acids in length.

### EXAMPLE 3

### Isolation of Bovine Serum Albumin Promoter/Enhancer

DNA is first isolated from the bovine liver (Wizard Genomic DNA Purification Kit, Promega, Madison, Wis.). PCR is then be used to amplify the serum albumin promoter region from the isolated DNA using two 40 base primers: (5° CTGGTGAAGATCTAGGGTTCTCATAACCTACAGAGAATT 3′; SEQ ID NO:5 and 5′ TCACCCACTCCTAGGTGCCAAAGTTTTGGGGTTGATAGAA 3′; SEQ ID NO:6). PCR amplification creates a promoter/enhancer fragment of ˜800 bp in length corresponding to the bovine serum albumin 5′ flanking region. The promoter fragment has an AvrII restriction site on the 3′ end of the gene and a Bgl II restriction site on the 5′ end of the gene, allowing easy attachment of the Apo E gene and the truncated LDL receptor gene to the promoter in the retroviral backbones. DNA sequencing is performed on all of the gene constructs to confirm that no mutations were introduced during the polymerase chain reaction and subsequent cloning.

### EXAMPLE 4

### Isolation of Bovine alpha-1-Antitrypsin Promoter/Enhancer

DNA is first isolated from the bovine liver (Wizard Genomic DNA Purification Kit, Promega, Madison, Wis.). PCR is then be used to amplify the alpha-1-antitrypsin promoter region from the isolated DNA using two primers: (5′CAAACGGGCTCGAGCCCACTCTGATCTCCCAGGGCGGCAGT3′; SEQ ID NO: 7 and 5′ ACAGTGCCAAGATCTATTCACTGTCCTAGGTCAGGGCT 3′; SEQ ID NO:8). PCR amplification creates a promoter/enhancer fragment of ˜400 bp in length corresponding to the bovine alpha-1-antitrypsin 5′ flanking region. The promoter fragment has an Bgl II restriction site on the 3′ end of the gene and a Xho I restriction site on the 5′ end of the gene, allowing easy attachment of the Apo E gene and the truncated LDL receptor gene to the promoter in the retroviral backbones. DNA sequencing is performed on all of the gene constructs to confirm that no mutations were introduced during the polymerase chain reaction and subsequent cloning.

### EXAMPLE 5

### Vector Construction

Upon isolation of the genes and the promoters described in Examples 1-4 above, they are cloned into a replication defective retroviral backbone. The backbone contains the Moloney Murine Leukemia Virus 5′ LTR, extended viral packaging signal, a multiple cloning site, an internal ribosome entry site, a RNA transport signal and the 3′ Moloney Murine Leukemia Virus 3′ LTR The RNA transport signal causes the mRNA for the gene to be transported from the nucleus to the cytoplasm and the internal ribosome entry site allow for more efficient ribosome attachment to the mRNA during translation. Four individual vector constructs are produced all in the same retroviral backbone. The four constructs are:

A. Bovine serum albumin promoter/enhancer-Bovine apoE gene

B. Bovine serum albumin promoter/enhancer-Truncated bovine LDL receptor gene

C. Bovine alpha-1-antitrypsin promoter/enhancer-Bovine apoE gene

D. Bovine alpha-1-antitrypsin promoter/enhancer-Truncated bovine LDL receptor gene

### EXAMPLE 6

### Packaging Cell Line Creation and Vector Propagation

The four gene constructs are next used for replication defective retroviral production. The expression of the fusogenic VSV G protein on the surface of cells results in syncytium formation and cell death. Therefore, in order to produce retroviral particles containing the VSV G protein as the membrane-associated protein a two-step approach was taken. First, stable cell lines expressing the gag and pol proteins from MoMLV at high levels are generated (e.g., 293GP^{SD} cells). The stable cell line which expresses the gag and pol proteins produces noninfectious viral particles lacking a membrane-associated protein (e.g., a envelope protein). The stable cell line is then co-transfected, using the calcium phosphate precipitation, with VSV-G and gene of interest plasmid DNAs. The pseudotyped vector generated is used to infect 293GP^{SD} cells to produce stably transformed cell lines. Stable cell lines can be transiently transfected with a plasmid capable of directing the high level expression of the VSV G protein (see below). The transiently transfected cells produce VSV G-pseudotyped retroviral vectors which can be collected from the cells over a period of 3 to 4 days before the producing cells die as a result of syncytium formation.

The first step in the production of VSV G-pseudotyped retroviral vectors, the generation of stable cell lines expressing the MoMLV gag and pol proteins is described below. The human adenovirus 5-transformed embryonal kidney cell line 293 (ATCC CRL 1573) is cotransfected with the pCMVgag-pol and the gene encoding for phleomycin. pCMV gag-pol contains the MoMLV gag and pol genes under the control of the CMV promoter (pCMV gag-pol is available from the ATCC).

The plasmid DNA us introduced into the 293 cells using calcium phosphate co-precipitation (Graham and Van der Eb, Virol. 52:456 [1973]). Approximately 5×10⁵ 293 cells are plated into a 100 mm tissue culture plate the day before the DNA co-precipitate is added. Stable transformants are selected by growth in DMEM-high glucose medium containing 10% FCS and 10 μg/ml phleomycin (selective medium). Colonies that grow in the selective medium are screened for extracellular reverse transcriptase activity (Goff et al., J. Virol. 38:239 [1981]) and intracellular p39gag expression. The presence of p30gag expression is determined by Western blotting using a goat-anti p30 antibody (NCI antiserum 77S000087). A clone which exhibited stable expression of the retroviral genes is selected. This clone is named 293GP^{SD} (293 gag-pol-San Diego). The 293GP^{SD} cell line, a derivative of the human Ad-5-transformed embryonal kidney cell line 293, was grown in DMEM-high glucose medium containing 10% FCS.

### EXAMPLE 7

### Preparation of Pseudotyped Retroviral Vectors Bearing the G Glycoprotein of VSV

In order to produce VSV G protein pseudotyped retrovirus the following steps are taken. The 293GP^{SD} cell line is co-transfected with VSV-G plasmid and DNA plasmid of interest. This co-transfection generates the infectious particles used to infect 293GP^{SD} cells to generate the packaging cell lines. This general method may be used to produce any of the vectors of the present invention.

### a) Cell Lines and Plasmids

The packaging cell line, 293GP^{SD} is grown in alpha-MEM-high glucose medium containing 10% FCS. The titer of the pseudo-typed virus may be determined using either 208F cells (Quade, Virol. 98:461 [1979]) or NIH/3T3 cells (ATCC CRL 1658); 208F and NIH/3T3 cells are grown in DMEM-high glucose medium containing 10% CS.

The plasmid pHCMV-G contains the VSV G gene under the transcriptional control of the human cytomegalovirus intermediate-early promoter (Yee et al., Meth. Cell Biol. 43:99 [1994]).

### b) Production of Stable Packaging Cell Lines, Pseudotyped Vector and Titering of Pseudotyped Vectors

One of the vectors of the present invention (e.g., SEQ ID NO:9) is co-transfected with pHCMV-G DNA into the packaging line 293GP^{SD} to produce virus. The resulting virus is then used to infect ₂₉₃GP^{SD} cells to transform the cells. The procedure for producing pseudotyped virus is carried out as described (Yee et al., Meth. Cell Biol. 43:99 [1994]. This is a retroviral gene construct that upon creation of infectious replication defective retroviral vector will cause the insertion of the sequence described above into the cells of interest.

Briefly, on day 1, approximately 5×10⁴ 293GP^{SD} cells are placed in a 75 cm² tissue culture flask. On the following day (day 2), the ₂₉₃GP^{SD} cells are transfected with 25 μg of plasmid DNA and 25 μg of VSV-G plasmid DNA using the standard calcium phosphate co-precipitation procedure (Graham and Van der Eb, Virol. 52:456 [1973]). A range of 10 to 40 μg of plasmid DNA may be used. Because 293GP^{SD} cells may take more than 24 hours to attach firmly to tissue culture plates, the 293GP^{SD} cells may be placed in 75 cm² flasks 48 hours prior to transfection. The transfected ₂₉₃GP^{SD} cells provide pseudotyped virus.

On day 3, approximately 1×10⁵ 293GP^{SD} cells are placed in a 75 cm² tissue culture flask 24 hours prior to the harvest of the pseudotyped virus from the transfected 293GP^{SD} cells. On day 4, culture medium is harvested from the transfected 2093GP^{SD} cells 48 hours after the application of the construct of interest and VSV-G DNA. The culture medium is filtered through a 0.45 μm filter and polybrene is added to a final concentration of 8 μg/ml. The culture medium containing virus is used to infect the 293GP^{SD} cells as follows. The culture medium is removed from the 293GP^{SD} cells and was replaced with the virus of interest (e.g., SEQ ID NO:9) containing culture medium. Polybrene is added to the medium following addition to cells. The virus containing medium is allowed to remain on the 293GP^{SD} cells for 24 hours. Following the 16 hour infection period (on day 5), the medium is removed from the 293GP^{SD} cells and replaced with fresh medium containing 400 μg/ml G418 (GIBCO/BRL). The medium is changed approximately every 3 days until G418-resistant colonies appear approximately two weeks later.

The G418-resistant 293 colonies are plated as single cells in 96 wells. Sixty to one hundred G418-resistant colonies are screened for the expression of the protein of interest (e.g., bovine apoE) in order to identify high producing clones. The top 10 clones in 96-well plates are transferred to 6-well plates and allowed to grow to confluency.

The top 10 clones are then expanded to screen for high titer production. Based on protein expression and titer production, 5 clonal cell lines are selected. One line is designated the master cell bank and the other 4 as backup cell lines. Pseudotyped vector is generated as follows. Approximately 1×10⁶ 293GPSD/virus of interest cells are placed into a 75cm² tissue culture flask. Twenty-four hours later, the cells are transfected with 25 μg of pHCMV-G plasmid DNA using calcium phosphate co-precipitation. Six to eight hours after the calcium-DNA precipitate is applied to the cells, the DNA solution is replaced with fresh culture medium (lacking G418). Longer transfection times (overnight) are found to result in the detachment of the majority of the 293GP^{SD}/virus of interest cells from the plate and are therefore avoided. The transfected 293GP^{SD}/virus of interest cells produce pseudotyped virus.

The pseudotyped virus generated from the transfected 293GP^{SD}/virus of interest cells can be collected at least once a day between 24 and 96 hr after transfection. The highest virus titer is generated approximately 48 to 72 hr after initial pHCMV-G transfection. While syncytium formation becomes visible about 48 hr after transfection in the majority of the transfected cells, the cells continue to generate pseudotyped virus for at least an additional 48 hr as long as the cells remained attached to the tissue culture plate. The collected culture medium containing the VSV G-pseudotyped virus is pooled, filtered through a 0.45 μm filter and stored at −80° C. or concentrated immediately and then stored at −80° C.

The titer of the VSV G-pseudotyped virus is then determined as follows. Approximately 5×10⁴ rat 208F fibroblasts cells are plated into 6 well plates. Twenty-fours hours after plating, the cells are infected with serial dilutions of the virus-containing culture medium in the presence of 8 μg/ml polybrene. Twenty four hours after infection with virus, the medium is replaced with fresh medium containing 400 μg/ml G418 and selection is continued for 14 days until G418-resistant colonies became visible. Viral titers are typically about 0.5 to 5.0×10⁶ colony forming units (cfu)/ml. The titer of the virus stock can be concentrated to a titer of greater than 10⁹ cfu/ml as described below.

### EXAMPLE 8

### Concentration of Pseudotyped Retroviral Vectors

The VSV G-pseudotyped virus is then concentrated to a high titer by one cycle of ultracentrifugation. However, two cycles can be performed for further concentration. The frozen culture medium collected as described in Example 7 which contains pseudotyped virus is thawed in a 37° C. water bath and then transferred to Oakridge centrifuge tubes (50 ml Oakridge tubes with sealing caps, Nalge Nunc International) previously sterilized by autoclaving. The virus is sedimented in a JA20 rotor (Beckman) at 48,000× g (20,000 rpm) at 4° C. for 120 min. The culture medium is then removed from the tubes in a biosafety hood and the media remaining in the tubes is aspirated to remove the supernatant. The virus pellet is resuspended to 0.5 to 1% of the original volume of culture medium DMEM. The resuspended virus pellet is incubated overnight at 4° C without swirling. The virus pellet is able to be dispersed with gentle pipetting after the overnight incubation without significant loss of infectious virus. The titer of the virus stock is routinely increased 100- to 300-fold after one round of ultracentrifugation. The efficiency of recovery of infectious virus varies between 30 and 100%.

The virus stock is then subjected to low speed centrifugation in a microfuge for 5 min at 4° C. to remove any visible cell debris or aggregated virions that are not resuspended under the above-conditions. It is noted that if the virus stock is not to be used for injection into oocytes or embryos, this centrifugation step may be omitted.

The virus stock can be subjected to another round of ultracentrifugation to further concentrate the virus stock. The resuspended virus from the first round of centrifugation is pooled and pelleted by a second round of ultracentrifugation which is performed as described above. Viral titers are increased approximately 2000-fold after the second round of ultracentrifugation (titers of the pseudotyped virus are typically greater than or equal to 1×10⁹ cfu/ml after the second round of ultracentrifugation).

The titers of the pre- and post-centrifugation fluids are determined by infection of 208F cells (NIH 3T3 or bovine mammary epithelial cells can also be employed) followed by selection of G418-resistant colonies as described above in Example 7.

### EXAMPLE 9

### Preparation of Pseudotyped Retrovirus For Injection of Gametes and Zygotes

The concentrated pseudotyped retroviruses are resuspended in 0.1× HBS (2.5 mM HEPES, pH 7.12, 14 mM NaCl, 75 μM Na2HPO4H2O) and 18 μl aliquots are placed in 0.5 ml vials (Eppendorf) and stored at −80° C. until use. The titer of the concentrated vector is determined by diluting 1 μl of the concentrated virus 10⁻⁷- or 10⁻⁸-fold with 0.1× HBS.

Gametes (pre-maturation and pre-fertilization oocytes) and zygotes (fertilized oocytes) are prepared and microinjected with retroviral stocks as described below.

### a) Solutions

Tyrodes-Lactate with HEPES (TL-HEPES): 114 mM NaCl, 3.2 mM KCl, 2.0 rM NaHCO₃, 0.4 mM Na₂H₂PO₄.H₂O, 10 mM Na-lactate, 2 mM CaCl₂.2H₂O, 0.5 mM MgCl₂.6H₂O, 10 mM HEPES, 100 IU/ml penicillin, 50 μg/ml phenol red, 1 mg/ml BSA fraction V, 0.2 mM pyruvate and 25 μg/ml gentamycin.

Maturation Medium: TC-199 medium (GIBCO) containing 10% FCS, 0.2 mM pyruvate, 5 μg/ml NIH o-LH (NIH), 25 μg/ml gentamycin and 1 μg/ml estradiol-17β.

Sperm-Tyrodes-Lactate (Sperm-TL): 100 mM NaCl, 3.2 mM KCl, 25 mM NaHCO₃, 0.29 mM Na₂H₂PO₄.H₂O, 21.6 mM Na-lactate, 2.1 mM CaCl₂.2H₂O, 0.4 mM MgCl₂.6H₂O, 10 mM HEPES, 50 μg/ml phenol red, 6 mg/ml BSA fraction V, 1.0 mM pyruvate and 25 μg/ml gentamycin.

Fertilization Medium: 114 mM NaCl, 3.2 mM KCl, 25 mM NaHCO₃, 0.4 mM Na₂H₂PO₄.H₂O, 10 mM Na-lactate, 2 mM CaCl₂.2H₂O, 0.5 mM MgCl₂.6H₂O, 100 IU/ml penicillin, 50 μg/ml phenol red, 6 mg/ml BSA fatty acid free, 0.2 mM pyruvate and 25 μg/ml gentamycin.

PHE: 1 mM hypotaurine, 2 mM penicillamine and 250 μM epinephrine.

Embryo Incubation+Amino Acids (EIAA): 114 μM NaCl, 3.2 μM KCl 25 μM NaHCO₃, 1.6 μg/ml L(+)-lactate, 10.7 μg/ml L-glutamine, 300 μg/ml BSA fatty acid free, 0.275 μg/ml pyruvate, 25 μg/ml gentamycin, 10 μl of 100× MEM amino acids stock (M7145, Sigma) per ml and 20 μl of 50× BME amino acids stock (B6766, Sigma) per ml.

0.1× HBS: 2.5 mM HEPES (pH 7.12), 14 mM NaCl and 75 μM Na₂HPO₄.H₂O.

### b) Preparation, Injection, Maturation and Fertilization of Pre-Maturation Oocytes

Oocytes are aspirated from small antral follicles on ovaries from dairy cattle obtained from a slaughterhouse. Freshly aspirated oocytes at the germinal vesicle (GV) stage, meiosis arrested, with the cumulus mass attached are selected (i.e., pre-maturation oocytes). The oocytes are then washed twice in freshly prepared TL-HEPES and transferred into a 100 μl drop of TL-HEPES for microinjection.

Concentrated retroviral particles (prepared as described in Example 8) are resuspended in 0.1× HBS, mixed with polybrene and loaded into the injection needle. Approximately 10 pl of the virus solution is then injected into the perivitelline space of pre-maturation oocytes.

Following injection, the pre-maturation oocytes are washed twice in fresh TL-HEPES and transferred into maturation medium (10 oocytes in 50 μl). The pre-maturation oocytes are then incubated in Maturation Medium for 24 hours at 37° C. which permits the oocytes to mature to the metaphase II stage. The matured oocytes are then washed twice in Sperm-TL and 10 oocytes are then transferred into 44 μl of Fertilization Medium. The mature oocytes (10 oocytes/44 μl Fertilization Medium) are then fertilized by the addition of 2 μl of sperm at a concentration of 2.5×10⁷/ml, 2 μl of PHE and 2 μl of heparin (fertilization mixture).

Sperm is prepared by discontinuous percoll gradient separation of frozen-thawed semen as described (Kim et al., Mol. Reprod. Develop., 35:105 [1993]). Briefly, percoll gradients are formed by placing 2 ml of each of 90% and 45% percoll in a 15 ml conical tube. Frozen-thawed semen is layered on top of the gradient and the tubes are centrifuged for 10 minutes at 700× g. Motile sperm are collected from the bottom of the tube.

The oocytes are incubated for 16 to 24 hours at 37° C. in the fertilization mixture. Following fertilization, the cumulus cells are removed by vortexing the cells (one cell stage zygotes, Pronucleus Stage) for 3 minutes to produce “nude” oocytes. The nude oocytes are then washed twice in embryo culture medium (EIAA) and 20 to 25 zygotes are then cultured in 50 μl drop of EIAA (without serum until Day 4 at which time the zygotes are placed in EIAA containing 10% serum) until the desired developmental stage was reached: approximately 48 hours or Day 2 (Day 0 is the day when the matured oocytes are co-cultured with sperm) for morula stage (8 cell stage) or Day 6-7 for blastocyst stage.

### c) Preparation, Injection and Fertilization of Pre-Fertilization Oocytes

Pre-maturation oocytes are harvested, washed twice with TL-HEPES as described above. The oocytes were then cultured in Maturation Medium (10 oocytes per 50 μl medium) for 16 to 20 hours to produce pre-fertilization oocytes (Metaphase II Stage). The pre-fertilization or matured oocytes are then vortexed for 3 minutes to remove the cumulus cells to produce nude oocytes. The nude oocytes are washed twice in TL-HEPES and then transferred into a 100 μl drop of TL-HEPES for microinjection. Microinjection is conducted as described above.

Following microinjection, the pre-fertilization oocytes are washed twice with TL-HEPES and then placed in Maturation Medium until fertilization. Fertilization is conducted as described above. Following fertilization, the zygotes are washed twice in EIAA and 20 to 25 zygotes were then cultured per 50 μl drop of EIAA until the desired developmental stage is reached.

### d) Preparation and Injection of One-Cell Stage Zygotes

Matured oocytes (Metaphase II stage) are generated as described above. The matured oocytes are then co-cultured in the presence of sperm for 16 to 20 hours as described above to generate zygotes at the pronucleus stage. Zygotes at the pronucleus stage are vortexed for 3 minutes to remove the cumulus cell layer prior to microinjection. Microinjection of retrovirus is conducted as described above. Following microinjection, the zygotes are washed four times in EIAA and then placed in an EIAA culture drop (25 zygotes per 50 μl drop of EIAA). The zygotes are cultured in EIAA (20 to 25 zygote per 50 μl drop of EIAA) until the desired developmental stage was reached.

### EXAMPLE 10

### Vector Evaluation

### A. Evaluation of Vectors in Hepatocytes

Mouse hepatocytes are prepared by collagenase perfusion and cultured in DME containing 10% fetal bovine serum. After 6 hours of culture, the cells are exposed to one of each of the pseudotyped retroviral vectors (as described above). Uninfected cell cultures prepared in parallel are used as a control of baseline expression.

The rate of apoB secretion is measured by quantitating the amount of ³⁵S-methionine/cysteine incorporated into immunoprecipitable protein. Albumin is also immunoprecipitated and used as a control for non-specific effects on protein synthesis and secretion. Immunoprecipitates are separated on SDS-PAGE and the apoB and albumin bands quantitated on a Phosphor-Imager.

Two types of analyses are performed. First, the overall rate of apoB secretion is determined in continuous incubations with the amino acid tracer to periods extending to 4 hours. Second, pulse-chase experiments are conducted; the tracer is present for only 7.5 minutes and is followed with a chase media containing a vast excess (10 mM) of unlabeled cysteine and methionine. This allows the estimation of the extent of post-translational degradation of apoB.

The pulse-chase experiment enables a mechanistic interpretation of the results. It determination of whether the soluble receptor and/or apoE affect the post-translational fate of apoB by changing the proportion of apoB subject to degradation. Although it is well-established that a decrease in apoB secretion leads to a decrease in triglyceride secretion and can lead to fatty liver, it does not necessarily follow that the converse is also true; i.e., if an increase in apoB secretion leads to increased triglyceride secretion and protection from fatty liver.

In order to determine if high-level expression of apoE or the soluble receptor reverses triglyceride accumulation subsequent to fatty acid supplementation hepatocytes are incubated with albumin-bound oleic acid at concentrations from 0 to 1.5 mM for 24 hours. This causes an approximate 3-fold increase in hepatocyte triglyceride stores. Then, they are infected with retroviral vectors carrying apoE, the soluble receptor, or uninfected and the cellular triglyceride mass and the mass of secreted triglyceride is measured.

It is next determined if high-level apoE or soluble receptor prevents triglyceride accumulation. It has been suggested that newly-synthesized triglyceride is the preferred substrate for VLDL triglyceride. Thus, it might be impossible to mobilize pre-stored triglyceride with increased apoB secretion. In these studies, cells are exposed to albumin-bound oleic acid at concentrations up to 1.5 mM. Cells are infected with the retroviral vectors for one hour, 24 hours prior to addition of oleic acid. The oleic acid is then added and after another 24 hours, the cellular triglyceride mass and mass of secreted triglyceride is measured.

### B. Evaluation of Retroviral Construct in 293 Cells

The apoE and truncated LDL receptor protein produced during the development of packaging cell lines are analyzed to confirm correct size and and a structure that is recognized by antibodies to the protein. The structure is confirmed by ELISA analysis of media samples and size is determined by denaturing PAGE-gel electrophoresis and western blotting.

### EXAMPLE 11

### Production of Transgenic Bovines

### A. Embryo Production

To produce the research evaluation group, oocytes are aspirated from ovaries collected fresh from a slaughterhouse that processes Holstein dairy cows. Large numbers of oocytes can be acquired and this permits several evaluations of vector integrity before a group of embryos are transferred to recipient dams. Alternatively, oocytes will be obtained by transvaginal aspiration directly from existing herds. Oocytes are aspirated and cultured 16-18 hours in oocyte maturation medium until vector injection into the perivitelline space is performed. Six to eight hours later the oocytes are fertilized in vitro with Holstein bull semen and further cultured for seven days. At this time healthy blastocysts are selected for transfer into recipient cows.

### B. Transfer, Gestation and Birth

Recipient mother cows are derived from an existing quarantined herd. The estrus cycles of these animals are synchronized to simulate a 7 day pregnancy, at this time the 7 day cultured embryo is transferred into the recipient. Embryo transfer is performed in accordance with standard commercial practices. The cattle are maintained under surveillance though the 280 day pregnancy.

### C. Evaluation of Offspring

Upon birth of the calves, blood and skin samples are collected to determine whether the animal is transgenic. DNA is isolated from blood and skin samples (Wizard Genomic DNA Purification Kit, Promega, Madison, Wis.). Transgenesis is determined using polymerase chain reaction. PCR is used to amplify a specific region of the transgene. Primers are used that will amplify a portion of the inserted DNA construct corresponding to the junction between the bovine serum albumin or bovine alpha-1-antitrypsin promoter and either the bovine Apo E gene or the bovine truncated LDL receptor gene. The PCR reactions are analyzed by 1% agarose gel electrophoresis. Positive animals exhibit a specific DNA band 500 bp in length. Negative animals exhibit no band.

### D. Metabolic Evaluation

Calves are raised to weaning at 8 weeks of age. Animals are then moved to loose housing and sample is collected for metabolic evaluation of gene functionality. Each group comprises both transgenic and non transgenic calves. If needed additional non-transgenic age matched calves are purchased from commercial sources to provide control animals. Two approaches are used to determine the effect on liver metabolism of the added transgene in young animals. These data provide an excellent early indicator of the propensity to accumulate fat in hepatocytes.

After weaning, calves are fed diets that exceed energy requirements for maintenance and growth for four weeks to increase fat depots. Control and transgenic calves are then feed restricted for 5 days to promote fat mobilization and increase fatty acid flux to the liver. After the initial 72 hours of fasting, Triton WR 1339 (0.4 g/kg body weight) is administered by IV injection. Triton inhibits very low density lipoprotein (VLDL) clearance from blood (Li et al., J. Lipid Res., 38:1277 [1997]). Intestinal VLDL production in fasted animals is negligible, therefore, hepatic export of triglyceride as VLDL can be estimated by monitoring the increase in blood triglyceride concentration over time. Blood samples are obtained prior to fasting, prior to Triton administration and periodically for 48 hours following. Blood samples are analyzed for glucose, beta-hydroxybutyrate (a ketone), nonesterified fatty acids, and triglyceride.

It is contemplated that transgenic animals will have a higher rate of hepatic triglyceride export and, therefore, a faster rate of triglyceride accumulation in blood. The protocol is performed on the five founder calves for each of the four gene constructs and for five control calves of similar age.

Hepatic triglyceride is monitored in control and transgenic animals that are used in the protocol described above. Liver samples are obtained by percutaneous needle biopsy prior to fasting and after 3 and 5 days of fasting. It is contemplated that transgenic calves will have lower hepatic triglyceride if rate of export has been enhanced.

A third evaluation is performed later in life. Ketosis and fatty liver typically manifest themselves following the second and later calvings. The transgenic LDL receptor and apoE heifers are maintained in the herd and followed through several pregnancies.

### E. Production of Elite Seedstock Animals

The group of elite oocytes are collected using ultrasound in vitro oocyte retrieval from a herd of elite embryo derived cattle. The same procedures described above for gene insertion into slaughterhouse oocytes are used for gene insertion into in vivo retrieved oocytes.

As described above, tissue samples (blood and skin snips) are collected from the transgenic bull and heifer calves derived from the elite genetic lines. The samples are evaluated by PCR for the presence of the transgenes. The animals are raised to puberty and evaluated using the metabolic indices described above. Semen is collected from the bulls and evaluated for transgene transmission by use of in vitro fertilization of non-transgenic oocytes. The presence of transgene in resultant embryos is determined by polymerase chain reaction. Heifers are superovulated, inseminated with non-transgenic semen and used to derive embryos for transfer to non-transgenic recipients. In founder animals where a single gene insertion has occurred in the oocyte, and which are thus heterozygotes, Mendelian inheritance is expected in the offspring, half of which will be transgenic.

### EXAMPLE 12

### Construction of Human/Bovine Vectors

### A. Human Albumin promoter/bovine ApoE

The Human Albumin promoter/bovine ApoE (SEQ ID NO:9) construct comprises the following elements, arranged in 5′ to 3′ order: Moloney Murine Sarcoma Virus 5′ LTR; Moloney Murine Leukemia Virus Extended Packaging Region; Neomycin Resistance Gene; Human Serum Albumin S° Flanking Region and Promoter; Double Mutated PPE Sequence; Bovine Apolipoprotein E cDNA; WPRE Sequence; Moloney Murine Leukemia Virus 3′ LTR.

This is a retroviral gene construct that upon creation of infectious replication defective retroviral vector will cause the insertion of a version of the sequence described above into the cells of interest. Upon insertion the human serum albumin regulatory sequences control the expression of the bovine apolipoprotein E gene. The PPE sequences and the WPRE sequence aid in moving the mRNA from the nucleus to the cytoplasm. The 3′ viral LTR provides the poly-adenylation sequence for the mRNA.

### B. Human Albumin promoter/Human truncated LDL Receptor

The Human Albumin promoter Human truncated LDL receptor (SEQ ID NO:10) construct comprises the following elements, arranged in 5′ to 3′ order: Moloney Murine Sarcoma Virus 5′ LTR; Moloney Murine Leukemia Virus Extended Packaging Region; Neomycin Resistance Gene; Human Serum Albumin 5° Flanking Region and Promoter; Double Mutated PPE Sequence; Human Truncated LDL Receptor cDNA ; WPRE Sequence; Moloney Murine Leukemia Virus 3′ LTR.

This is a retroviral gene construct that upon creation of infectious replication defective retroviral vector will cause the insertion of a version of the sequence described above into the cells of interest. Upon insertion the human serum albumin regulatory sequences control the expression of the human truncated LDL receptor gene. The PPE sequences and the WPRE sequence aid in moving the mRNA from the nucleus to the cytoplasm. The 3′ viral LTR provides the poly-adenylation sequence for the mRNA.

### C. Human alpha-1-antitrypsin promoter/Bovine ApoE

The Human alpha-1-antitrypsin promoter/Bovine ApoE (SEQ ID NO:11) construct comprises the following elements, arranged in 5′ to 3′ order: Moloney Murine Sarcoma Virus 5′ LTR; Moloney Murine Leukemia Virus Extended Packaging Region; Neomycin Resistance Gene; Human Alpha-1-Antitrypsin 5° Flanking Region and Promoter; Double Mutated PPE Sequence; Bovine Apolipoprotein E cDNA; WPRE Sequence; Moloney Murine Leukemia Virus 3′ LTR

This is a retroviral gene construct that upon creation of infectious replication defective retroviral vector will cause the insertion of a version of the sequence described above into the cells of interest. Upon insertion the human alpha-1-antitrypsin regulatory sequences control the expression of the bovine apolipoprotein E. The PPE sequences and the WPRE sequence aid in moving the mRNA from the nucleus to the cytoplasm. The 3′ viral LTR provides the poly-adenylation sequence for the mRNA.

### D. Human alpha-1-antitrypsin/Human truncated LDL

The Human alpha-1-antitrypsin promoter/Human truncated LDL receptor (SEQ ID NO:12) construct comprises the following elements, arranged in 5′ to 3′ order: Moloney Murine Sarcoma Virus 5′ LTR; Moloney Murine Leukemia Virus Extended Packaging Region; Neomycin Resistance Gene; Human Alpha-1-Antitrypsin 5′ Flanking Region and Promoter; Double Mutated PPE Sequence; Human Truncated LDL Receptor cDNA ; WPRE Sequence; Moloney Murine Leukemia Virus 3′ LTR This is a retroviral gene construct that upon creation of infectious replication defective retroviral vector will cause the insertion of a version of the sequence described above into the cells of interest. Upon insertion the human alpha-1-antitrypsin regulatory sequences control the expression of the human truncated LDL receptor cDNA. The PPE sequences and the WPRE sequence aid in moving the mRNA from the nucleus to the cytoplasm. The 3′ viral LTR provides the poly-adenylation sequence for the mRNA.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology, developmental biology, biochemistry, or related fields are intended to be within the scope of the following claims.

What is claimed is:

1. A method of producing a fatty liver disease resistant transgenic bovine, comprising:
a. providing
i) a gene construct comprising a liver-specific promoter operably linked to a gene of interest selected from the group consisting of ApoE and truncated soluble LDL receptor;
ii) a cell selected from the group consisting of oocytes and zygotes; and
iii) a bovine;
b. introducing said gene construct into said cell; and
c. transplanting said cell into said bovine to generate a transgenic bovine, wherein said transgenic bovine has increased lipid mobility as compared to a non-transgenic bovine.

2. The method of claim 1, wherein said promoter is a bovine albumin promoter.

3. The method of claim 2, wherein said bovine albumin promoter comprises a promoter selected from the group consisting of SEQ ID NO:13 and sequences hybridizable to SEQ ID NO:13 under conditions of low to high stringency.

4. The method of claim 1, wherein said promoter is a bovine alpha-1-antitrypsin promoter.

5. The method of claim 4, wherein said bovine alpha-1-antitrypsin promoter comprises a promoter selected from the group consisting of SEQ ID NO:14 and sequences hybridizable to SEQ ID NO: 14 under conditions of low to high stringency.

6. The method of claim 1, wherein said ApoE gene comprises SEQ ID NO:15

7. The method of claim 1, wherein said ApoE gene comprises a sequence selected from the group consisting of SEQ ID NO:15 and sequences hybridizable to SEQ ID NO:15 under conditions of low to high stringency.

8. The method of claim 1, wherein said bovine truncated soluble LDL receptor gene comprises a sequence selected from the group consisting of SEQ ID NO:16 and sequences hybridizable to SEQ ID NO:16 under conditions of low to high stringency.

9. The method of claim 1, wherein said cell is an oocyte.

10. A transgenic bovine produced by the method of claim 1.

11. The transgenic bovine of claim 10, wherein said bovine has increased resistance to fatty liver disease as compared to a non-transgenic bovine.

12. The transgenic bovine of claim 10, wherein said bovine has an increased level of apoB protein secretion as compared to a non-transgenic bovine.

13. The transgenic bovine of claim 10, wherein said bovine has increased lipid mobility as compared to a non-transgenic bovine.

14. A fatty liver disease resistant bovine comprising a transgene; wherein said transgene comprises an ApoE gene operably linked to a liver-specific promoter selected from the group consisting of an alpha-1-antitrypsin promoter and an albumin promoter.

15. The bovine of claim 14, wherein said bovine has increased resistance to fatty liver disease as compared to a non-transgenic bovine.

16. The bovine of claim 14, wherein said bovine has an increased level of apoB protein secretion as compared to a non-transgenic bovine.

17. The bovine of claim 14, wherein said bovine has increased lipid mobility as compared to a non-transgenic bovine.

18. The bovine of claim 14, wherein said bovine ApoE gene comprises SEQ ID NO:15

19. The bovine of claim 14, wherein said ApoE gene comprises a sequence selected from the group consisting of SEQ ID NO:15 and sequences hybridizable to SEQ ID NO:15 under conditions of low to high stringency.

20. A fatty liver disease resistant bovine comprising a transgene; wherein said transgene comprises a truncated soluble LDL receptor gene operably linked to a liver-specific promoter selected from the group consisting of an alpha-1-antitrypsin promoter and an albumin promoter.

21. The bovine of claim 20, wherein said bovine has increased resistance to fatty liver disease as compared to a non-transgenic bovine.

22. The bovine of claim 20, wherein said bovine has an increased level of apoB protein secretion as compared to a non-transgenic bovine.

23. The bovine of claim 20, wherein said bovine has increased lipid mobility as compared to a non-transgenic bovine.

24. The bovine of claim 20, wherein said truncated soluble LDL receptor gene comprises a sequence selected from the group consisting of SEQ ID NO:16 and sequences hybridizable to SEQ ID NO:16 under conditions of low to high stringency.

25. A method of modifying the metabolism of a bovine comprising:
a) providing:
i) a bovine oocyte, embryo, or zygote; and
ii) an exogenous gene construct encoding a gene that regulates metabolism; and
b) introducing said exogenous gene construct into said bovine oocyte, embryo, or zygote, so that the metabolism of the resulting transgenic animal is altered as compared to non-transgenic animals.

26. The transgenic bovine produced by the method of claim 25.

27. A transgenic bovine having a genome, said genome comprising an exogenous gene construct encoding a gene that regulates metabolism, wherein said metabolism of said transgenic bovine is altered as compared to nontransgenic bovines.

28. A nucleic acid construct comprising a liver specific promoter operably linked to a gene of interest selected from the group consisting of ApoE and truncated soluble LDL receptor.

29. The nucleic acid construct of claim 28, wherein said gene of interest is ApoE.

30. The nucleic acid construct of claim 29, wherein said ApoE gene is selected from the group consisting of SEQ ID NO:15 and sequences hybridizable to SEQ ID NO:15 under conditions of low to high stringency.

31. The nucleic acid construct of claim 28, wherein said gene of interest is truncated soluble LDL receptor.

32. The nucleic acid construct of claim 31, wherein said truncated soluble LDL receptor gene is selected from the group consisting of SEQ ID NO:16 and sequences hybridizable to SEQ ID NO:16 under conditions of low to high stringency.

33. The nucleic acid construct of claim 28, wherein said liver specific promoter is the bovine alpha-1-antitrypsin promoter.

34. The nucleic acid construct of claim 33, wherein said bovine alpha-1-antitrypsin promoter is selected from the group consisting of SEQ ID NO:14 and sequences hybridizable to SEQ ID NO: 14 under conditions of low to high stringency.

35. The nucleic acid construct of claim 28, further comprising retroviral elements.

36. A bovine comprising the nucleic acid construct of claim 28.

37. A composition or method as substantially described herein in any of the examples.


SEQUENCE LISTING (16 sequences)

SEQ ID NO: 1 - DNA, 39 bp, Artificial Sequence
  Synthetic
gcggttggcctagggcaagccagaagatgaaggttctgt

SEQ ID NO: 2 - DNA, 40 bp, Artificial Sequence
  Synthetic
ggcgatgcgtcgacgctcaatgattctcactgggcggaga

SEQ ID NO: 3 - DNA, 40 bp, Artificial Sequence
  Synthetic
cgggacactcctaggcagaggctgcgagcatggggccctg

SEQ ID NO: 4 - DNA, 41 bp, Artificial Sequence
  Synthetic
ctgtcactcgtcgactcaatcttcgcatcttcgctgggcca

SEQ ID NO: 5 - DNA, 39 bp, Artificial Sequence
  Synthetic
ctggtgaagatctagggttctcataacctacagagaatt

SEQ ID NO: 6 - DNA, 40 bp, Artificial Sequence
  Synthetic
tcacccactcctaggtgccaaagttttggggttgatagaa

SEQ ID NO: 7 - DNA, 41 bp, Artificial Sequence
  Synthetic
caaacgggctcgagcccactctgatctcccagggcggcagt

SEQ ID NO: 8 - DNA, 38 bp, Artificial Sequence
  Synthetic
acagtgccaagatctattcactgtcctaggtcagggct

SEQ ID NO: 9 - DNA, 6026 bp, Artificial Sequence
  Synthetic
tttgaaagaccccacccgtaggtggcaagctagcttaagtaacgccactttgcaaggcatggaaaaatacataactgagaatagaaaagttcagatcaaggtcaggaacaaagaaacagctgaataccaaacaggatatctgtggtaagcggttcctgccccggctcagggccaagaacagatgagacagctgagtgatgggccaaacaggatatctgtggtaagcagttcctgccccggctcggggccaagaacagatggtccccagatgcggtccagccctcagcagtttctagtgaatcatcagatgtttccagggtgccccaaggacctgaaaatgaccctgtaccttatttgaactaaccaatcagttcgcttctcgcttctgttcgcgcgcttccgctctccgagctcaataaaagagcccacaacccctcactcggcgcgccagtcttccgatagactgcgtcgcccgggtacccgtattcccaataaagcctcttgctgtttgcatccgaatcgtggtctcgctgttccttgggagggtctcctctgagtgattgactacccacgacgggggtctttcatttgggggctcgtccgggatttggagacccctgcccagggaccaccgacccaccaccgggaggtaagctggccagcaacttatctgtgtctgtccgattgtctagtgtctatgtttgatgttatgcgcctgcgtctgtactagttagctaactagctctgtatctggcggacccgtggtggaactgacgagttctgaacacccggccgcaaccctgggagacgtcccagggactttgggggccgtttttgtggcccgacctgaggaagggagtcgatgtggaatccgaccccgtcaggatatgtggttctggtaggagacgagaacctaaaacagttcccgcctccgtctgaatttttgctttcggtttggaaccgaagccgcgcgtcttgtctgctgcagcgctgcagcatcgttctgtgttgtctctgtctgactgtgtttctgtatttgtctgaaaattagggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccggaattccgatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccgggctcgatcccctcgcgagttggttcagctgctgcctgaggctggacgacctcgcggagttctaccggcagtgcaaatccgtcggcatccaggaaaccagcagcggctatccgcgcatccatgcccccgaactgcaggagtggggaggcacgatggccgctttggtcgaggcggatctagggttctcataacctacagagaatttggggtcagcctgtcctattgtatattatggcaaagataatcatcatctcatttgggtccattttcctctccatctctgcttaactgaagatcccatgagatatactcacactgaatctaaatagcctatctcagggcttgaatcacatgtgggccacagcaggaatgggaacatggaatttctaagtcctatcttacttgttattgttgctatgtctttttcttagtttgcatctgaggcaacatcagctttttcagacagaatggctttggaatagtaaaaaagacacagaagccctaaaatatgtatgtatgtatatgtgtgtgtgcatgcgtgagtacttgtgtgtaaatttttcattatctataggtaaaagcacacttggaattagcaatagatgcaatttgggacttaactctttcagtatgtcttatttctaagcaaagtatttagtttggttagtaattactaaacactgagaactaaattgcaaacaccaagaactaaaatgttcaagtgggaaattacagttaaataccatggtaatgaataaaaggtacaaatcgtttaaactcttatgtaaaatttgataagatgttttacacaactttaatacattgacaaggtcttgtggagaaaacagttccagatggtaaatatacacaagggatttagtcaaacaattttttggcaagaatattatgaattttgtaatcggttggcagccaatgaaatacaaagatgagtctagttaataatctacaattattggttaaagaagtatattagtgctaatttccctccgtttgtcctagcttttctcttctgtcaaccccacacgcctttggcaagatccgattacttactggcaggtgctgggggcttccgagacaatcgcgaacatctacaccacacaacaccgcctcgaccagggtgagatatcggccggggacgcggcggtggtaattacaagcgagatccgattacttactggcaggtgctgggggcttccgagacaatcgcgaacatctacaccacacaacaccgcctcgaccagggtgagatatcggccggggacgcggcggtggtaattacaagcgagatctcgagttaacagatctaggcctcctagggcaagccagaagatgaaggttctgtgggttgccgtggtggtcgcgcttctggcaggatgccaggcggatatggagggagagctggggcccgaggagcccctgactacgcagcagccccgggggaaggacagccagccttgggagcaggcgctgggccgcttctgggattacctgcgctgggtgcagaccctgtctgaccaggtgcaggaggagctgctcaacacccaggtcattcaggaactgacggcgctgatggaggagaccatgaaggaggtgaaggcctacaaggaggagctggagggacagctaggccccatggcccaggagacacaggcccgcgtgtccaaggagctgcaggcagcccaggcccggctaggctccgacatggaggacttgtgcggacgcctggcacagtaccgaagcgaggtgcaggccatgctgggccagtctaccgaggagctgcgggcccgcatggcctcccacctgcgcaagctgccgaagcggctgctccgcgacgctgacgacctgaagaagcgcctggccgtctaccaagctggggccagcgagggtgccgagcgcagcttgagcgccatccgcgagcgcttcgggcccctggtggagcagggccaatcgcgggcagccaccctgagcaccctggccggccagccgctgctggagcgtgccgaggcctggcgccagaagctgcacgggcgcctggaggaggtgggcgtccgggcccaggaccgcctggataagatacgccagcagctagaggaggtgcacgccaaggtcgaggagcagggcaaccagatgcgcctgcaggccgaggcattccaggcccgcctcaggagctggttcgagcccctggtggaagacatgcagcgccagtgggctgggctggtggagaaggtgcagttggctctgcgccccagccccacctctccgcccagtgagaatcattgagcgtcgacatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgatcgataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatt

SEQ ID NO: 10 - DNA, 6140 bp, Artificial Sequence
  Synthetic
tttgaaagaccccacccgtaggtggcaagctagcttaagtaacgccactttgcaaggcatggaaaaatacataactgagaatagaaaagttcagatcaaggtcaggaacaaagaaacagctgaataccaaacaggatatctgtggtaagcggttcctgccccggctcagggccaagaacagatgagacagctgagtgatgggccaaacaggatatctgtggtaagcagttcctgccccggctcggggccaagaacagatggtccccagatgcggtccagccctcagcagtttctagtgaatcatcagatgtttccagggtgccccaaggacctgaaaatgaccctgtaccttatttgaactaaccaatcagttcgcttctcgcttctgttcgcgcgcttccgctctccgagctcaataaaagagcccacaacccctcactcggcgcgccagtcttccgatagactgcgtcgcccgggtacccgtattcccaataaagcctcttgctgtttgcatccgaatcgtggtctcgctgttccttgggagggtctcctctgagtgattgactacccacgacgggggtctttcatttgggggctcgtccgggatttggagacccctgcccagggaccaccgacccaccaccgggaggtaagctggccagcaacttatctgtgtctgtccgattgtctagtgtctatgtttgatgttatgcgcctgcgtctgtactagttagctaactagctctgtatctggcggacccgtggtggaactgacgagttctgaacacccggccgcaaccctgggagacgtcccagggactttgggggccgtttttgtggcccgacctgaggaagggagtcgatgtggaatccgaccccgtcaggatatgtggttctggtaggagacgagaacctaaaacagttcccgcctccgtctgaatttttgctttcggtttggaaccgaagccgcgcgtcttgtctgctgcagcgctgcagcatcgttctgtgttgtctctgtctgactgtgtttctgtatttgtctgaaaattagggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccggaattccgatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccgggctcgatcccctcgcgagttggttcagctgctgcctgaggctggacgacctcgcggagttctaccggcagtgcaaatccgtcggcatccaggaaaccagcagcggctatccgcgcatccatgcccccgaactgcaggagtggggaggcacgatggccgctttggtcgaggcggatctagggttctcataacctacagagaatttggggtcagcctgtcctattgtatattatggcaaagataatcatcatctcatttgggtccattttcctctccatctctgcttaactgaagatcccatgagatatactcacactgaatctaaatagcctatctcagggcttgaatcacatgtgggccacagcaggaatgggaacatggaatttctaagtcctatcttacttgttattgttgctatgtctttttcttagtttgcatctgaggcaacatcagctttttcagacagaatggctttggaatagtaaaaaagacacagaagccctaaaatatgtatgtatgtatatgtgtgtgtgcatgcgtgagtacttgtgtgtaaatttttcattatctataggtaaaagcacacttggaattagcaatagatgcaatttgggacttaactctttcagtatgtcttatttctaagcaaagtatttagtttggttagtaattactaaacactgagaactaaattgcaaacaccaagaactaaaatgttcaagtgggaaattacagttaaataccatggtaatgaataaaaggtacaaatcgtttaaactcttatgtaaaatttgataagatgttttacacaactttaatacattgacaaggtcttgtggagaaaacagttccagatggtaaatatacacaagggatttagtcaaacaattttttggcaagaatattatgaattttgtaatcggttggcagccaatgaaatacaaagatgagtctagttaataatctacaattattggttaaagaagtatattagtgctaatttccctccgtttgtcctagcttttctcttctgtcaaccccacacgcctttggcaagatccgattacttactggcaggtgctgggggcttccgagacaatcgcgaacatctacaccacacaacaccgcctcgaccagggtgagatatcggccggggacgcggcggtggtaattacaagcgagatccgattacttactggcaggtgctgggggcttccgagacaatcgcgaacatctacaccacacaacaccgcctcgaccagggtgagatatcggccggggacgcggcggtggtaattacaagcgagatctcgagttaacagatctaggcctcctaggcagaggctgcgagcatggggccctggggctggaaattgcgctggaccgtcgccttgctcctcgccgcggcggggactgcagtgggcgacagatgtgaaagaaacgagttccagtgccaagacgggaaatgcatctcctacaagtgggtctgcgatggcagcgctgagtgccaggatggctctgatgagtcccaggagacgtgcttgtctgtcacctgcaaatccggggacttcagctgtgggggccgtgtcaaccgctgcattcctcagttctggaggtgcgatggccaagtggactgcgacaacggctcagacgagcaaggctgtccccccaagacgtgctcccaggacgagtttcgctgccacgatgggaagtgcatctctcggcagttcgtctgtgactcagaccgggactgcttggacggctcagacgaggcctcctgcccggtgctcacctgtggtcccgccagcttccagtgcaacagctccacctgcatcccccagctgtgggcctgcgacaacgaccccgactgcgaagatggctcggatgagtggccgcagcgctgtaggggtctttacgtgttccaaggggacagtagcccctgctcggccttcgagttccactgcctaagtggcgagtgcatccactccagctggcgctgtgatggtggccccgactgcaaggacaaatctgacgaggaaaactgcgctgtggccacctgtcgccctgacgaattccagtgctctgatggaaactgcatccatggcagccggcagtgtgaccgggaatatgactgcaaggacatgagcgatgaagttggctgcgttaatgtgacactctgcgagggacccaacaagttcaagtgtcacagcggcgaatgcatcaccctggacaaagtctgcaacatggctagagactgccgggactggtcagatgaacccatcaaagagtgcgggaccaacgaatgcttggacaacaacggcggctgttcccacgtctgcaatgaccttaagatcggctacgagtgcctgtgccccgacggcttccagctggtggcccagcgaagatgcgaagattgagtcgacatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgatcgataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatt

SEQ ID NO: 11 - DNA, 5617 bp, Artificial Sequence
  Synthetic
tttgaaagaccccacccgtaggtggcaagctagcttaagtaacgccactttgcaaggcatggaaaaatacataactgagaatagaaaagttcagatcaaggtcaggaacaaagaaacagctgaataccaaacaggatatctgtggtaagcggttcctgccccggctcagggccaagaacagatgagacagctgagtgatgggccaaacaggatatctgtggtaagcagttcctgccccggctcggggccaagaacagatggtccccagatgcggtccagccctcagcagtttctagtgaatcatcagatgtttccagggtgccccaaggacctgaaaatgaccctgtaccttatttgaactaaccaatcagttcgcttctcgcttctgttcgcgcgcttccgctctccgagctcaataaaagagcccacaacccctcactcggcgcgccagtcttccgatagactgcgtcgcccgggtacccgtattcccaataaagcctcttgctgtttgcatccgaatcgtggtctcgctgttccttgggagggtctcctctgagtgattgactacccacgacgggggtctttcatttgggggctcgtccgggatttggagacccctgcccagggaccaccgacccaccaccgggaggtaagctggccagcaacttatctgtgtctgtccgattgtctagtgtctatgtttgatgttatgcgcctgcgtctgtactagttagctaactagctctgtatctggcggacccgtggtggaactgacgagttctgaacacccggccgcaaccctgggagacgtcccagggactttgggggccgtttttgtggcccgacctgaggaagggagtcgatgtggaatccgaccccgtcaggatatgtggttctggtaggagacgagaacctaaaacagttcccgcctccgtctgaatttttgctttcggtttggaaccgaagccgcgcgtcttgtctgctgcagcgctgcagcatcgttctgtgttgtctctgtctgactgtgtttctgtatttgtctgaaaattagggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccggaattccgatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccgggctcgatcccctcgcgagttggttcagctgctgcctgaggctggacgacctcgcggagttctaccggcagtgcaaatccgtcggcatccaggaaaccagcagcggctatccgcgcatccatgcccccgaactgcaggagtggggaggcacgatggccgctttggtcgaggcggatctcccactctgatctcccagggcggcagtaagtcttcagcatcaggcattttggggtgactcagtaaatggtagatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatagatccgattacttactggcaggtgctgggggcttccgagacaatcgcgaacatctacaccacacaacaccgcctcgaccagggtgagatatcggccggggacgcggcggtggtaattacaagcgagatccgattacttactggcaggtgctgggggcttccgagacaatcgcgaacatctacaccacacaacaccgcctcgaccagggtgagatatcggccggggacgcggcggtggtaattacaagcgagatctcgagttaacagatctaggcctcctagggcaagccagaagatgaaggttctgtgggttgccgtggtggtcgcgcttctggcaggatgccaggcggatatggagggagagctggggcccgaggagcccctgactacgcagcagccccgggggaaggacagccagccttgggagcaggcgctgggccgcttctgggattacctgcgctgggtgcagaccctgtctgaccaggtgcaggaggagctgctcaacacccaggtcattcaggaactgacggcgctgatggaggagaccatgaaggaggtgaaggcctacaaggaggagctggagggacagctaggccccatggcccaggagacacaggcccgcgtgtccaaggagctgcaggcagcccaggcccggctaggctccgacatggaggacttgtgcggacgcctggcacagtaccgaagcgaggtgcaggccatgctgggccagtctaccgaggagctgcgggcccgcatggcctcccacctgcgcaagctgccgaagcggctgctccgcgacgctgacgacctgaagaagcgcctggccgtctaccaagctggggccagcgagggtgccgagcgcagcttgagcgccatccgcgagcgcttcgggcccctggtggagcagggccaatcgcgggcagccaccctgagcaccctggccggccagccgctgctggagcgtgccgaggcctggcgccagaagctgcacgggcgcctggaggaggtgggcgtccgggcccaggaccgcctggataagatacgccagcagctagaggaggtgcacgccaaggtcgaggagcagggcaaccagatgcgcctgcaggccgaggcattccaggcccgcctcaggagctggttcgagcccctggtggaagacatgcagcgccagtgggctgggctggtggagaaggtgcagttggctctgcgccccagccccacctctccgcccagtgagaatcattgagcgtcgacatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgatcgataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatt

SEQ ID NO: 12 - DNA, 5731 bp, Artificial Sequence
  Synthetic
tttgaaagaccccacccgtaggtggcaagctagcttaagtaacgccactttgcaaggcatggaaaaatacataactgagaatagaaaagttcagatcaaggtcaggaacaaagaaacagctgaataccaaacaggatatctgtggtaagcggttcctgccccggctcagggccaagaacagatgagacagctgagtgatgggccaaacaggatatctgtggtaagcagttcctgccccggctcggggccaagaacagatggtccccagatgcggtccagccctcagcagtttctagtgaatcatcagatgtttccagggtgccccaaggacctgaaaatgaccctgtaccttatttgaactaaccaatcagttcgcttctcgcttctgttcgcgcgcttccgctctccgagctcaataaaagagcccacaacccctcactcggcgcgccagtcttccgatagactgcgtcgcccgggtacccgtattcccaataaagcctcttgctgtttgcatccgaatcgtggtctcgctgttccttgggagggtctcctctgagtgattgactacccacgacgggggtctttcatttgggggctcgtccgggatttggagacccctgcccagggaccaccgacccaccaccgggaggtaagctggccagcaacttatctgtgtctgtccgattgtctagtgtctatgtttgatgttatgcgcctgcgtctgtactagttagctaactagctctgtatctggcggacccgtggtggaactgacgagttctgaacacccggccgcaaccctgggagacgtcccagggactttgggggccgtttttgtggcccgacctgaggaagggagtcgatgtggaatccgaccccgtcaggatatgtggttctggtaggagacgagaacctaaaacagttcccgcctccgtctgaatttttgctttcggtttggaaccgaagccgcgcgtcttgtctgctgcagcgctgcagcatcgttctgtgttgtctctgtctgactgtgtttctgtatttgtctgaaaattagggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccggaattccgatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccgggctcgatcccctcgcgagttggttcagctgctgcctgaggctggacgacctcgcggagttctaccggcagtgcaaatccgtcggcatccaggaaaccagcagcggctatccgcgcatccatgcccccgaactgcaggagtggggaggcacgatggccgctttggtcgaggcggatctcccactctgatctcccagggcggcagtaagtcttcagcatcaggcattttggggtgactcagtaaatggtagatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatagatccgattacttactggcaggtgctgggggcttccgagacaatcgcgaacatctacaccacacaacaccgcctcgaccagggtgagatatcggccggggacgcggcggtggtaattacaagcgagatccgattacttactggcaggtgctgggggcttccgagacaatcgcgaacatctacaccacacaacaccgcctcgaccagggtgagatatcggccggggacgcggcggtggtaattacaagcgagatctcgagttaacagatctaggcctcctaggcagaggctgcgagcatggggccctggggctggaaattgcgctggaccgtcgccttgctcctcgccgcggcggggactgcagtgggcgacagatgtgaaagaaacgagttccagtgccaagacgggaaatgcatctcctacaagtgggtctgcgatggcagcgctgagtgccaggatggctctgatgagtcccaggagacgtgcttgtctgtcacctgcaaatccggggacttcagctgtgggggccgtgtcaaccgctgcattcctcagttctggaggtgcgatggccaagtggactgcgacaacggctcagacgagcaaggctgtccccccaagacgtgctcccaggacgagtttcgctgccacgatgggaagtgcatctctcggcagttcgtctgtgactcagaccgggactgcttggacggctcagacgaggcctcctgcccggtgctcacctgtggtcccgccagcttccagtgcaacagctccacctgcatcccccagctgtgggcctgcgacaacgaccccgactgcgaagatggctcggatgagtggccgcagcgctgtaggggtctttacgtgttccaaggggacagtagcccctgctcggccttcgagttccactgcctaagtggcgagtgcatccactccagctggcgctgtgatggtggccccgactgcaaggacaaatctgacgaggaaaactgcgctgtggccacctgtcgccctgacgaattccagtgctctgatggaaactgcatccatggcagccggcagtgtgaccgggaatatgactgcaaggacatgagcgatgaagttggctgcgttaatgtgacactctgcgagggacccaacaagttcaagtgtcacagcggcgaatgcatcaccctggacaaagtctgcaacatggctagagactgccgggactggtcagatgaacccatcaaagagtgcgggaccaacgaatgcttggacaacaacggcggctgttcccacgtctgcaatgaccttaagatcggctacgagtgcctgtgccccgacggcttccagctggtggcccagcgaagatgcgaagattgagtcgacatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgatcgataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatt

SEQ ID NO: 13 - DNA, 877 bp, Bos taurus
gggttctcataacctacagagaatttggggtcagcctgtcctattgtatattatggcaaagataatcatcatctcatttgggtccattttcctctccatctctgcttaactgaagatcccatgagatatactcacactgaatctaaatagcctatctcagggcttgaatcacatgtgggccacagcaggaatgggaacatggaatttctaagtcctatcttacttgttattgttgctatgtctttttcttagtttgcatctgaggcaacatcagctttttcagacagaatggctttggaatagtaaaaaagacacagaagccctaaaatatgtatgtatgtatatgtgtgtgtgcatgcgtgagtacttgtgtgtaaatttttcattatctataggtaaaagcacacttggaattagcaatagatgcaatttgggacttaactctttcagtatgtcttatttctaagcaaagtatttagtttggttagtaattactaaacactgagaactaaattgcaaacaccaagaactaaaatgttcaagtgggaaattacagttaaataccatggtaatgaataaaaggtacaaatcgtttaaactcttatgtaaaatttgataagatgttttacacaactttaatacattgacaaggtcttgtggagaaaacagttccagatggtaaatatacacaagggatttagtcaaacaattttttggcaagaatattatgaattttgtaatcggttggcagccaatgaaatacaaagatgagtctagttaataatctacaattattggttaaagaagtatattagtgctaatttccctccgtttgtcctagcttttctcttctgtcaaccccacacgcctttggcaa

SEQ ID NO: 14 - DNA, 467 bp, Bos taurus
ccactctgatctcccagggcggcagtaagtcttcagcatcaggcattttggggtgactcagtaaatggtagatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaat

SEQ ID NO: 15 - DNA, 965 bp, Bos taurus
gcaagccagaagatgaaggttctgtgggttgccgtggtggtcgcgcttctggcaggatgccaggcggatatggagggagagctggggcccgaggagcccctgactacgcagcagccccgggggaaggacagccagccttgggagcaggcgctgggccgcttctgggattacctgcgctgggtgcagaccctgtctgaccaggtgcaggaggagctgctcaacacccaggtcattcaggaactgacggcgctgatggaggagaccatgaaggaggtgaaggcctacaaggaggagctggagggacagctaggccccatggcccaggagacacaggcccgcgtgtccaaggagctgcaggcagcccaggcccggctaggctccgacatggaggacttgtgcggacgcctggcacagtaccgaagcgaggtgcaggccatgctgggccagtctaccgaggagctgcgggcccgcatggcctcccacctgcgcaagctgccgaagcggctgctccgcgacgctgacgacctgaagaagcgcctggccgtctaccaagctggggccagcgagggtgccgagcgcagcttgagcgccatccgcgagcgcttcgggcccctggtggagcagggccaatcgcgggcagccaccctgagcaccctggccggccagccgctgctggagcgtgccgaggcctggcgccagaagctgcacgggcgcctggaggaggtgggcgtccgggcccaggaccgcctggataagatacgccagcagctagaggaggtgcacgccaaggtcgaggagcagggcaaccagatgcgcctgcaggccgaggcattccaggcccgcctcaggagctggttcgagcccctggtggaagacatgcagcgccagtgggctgggctggtggagaaggtgcagttggctctgcgccccagccccacctctccgcccagtgagaatcattgagc

SEQ ID NO: 16 - DNA, 1079 bp, Bos taurus
cagaggctgcgagcatggggccctggggctggaaattgcgctggaccgtcgccttgctcctcgccgcggcggggactgcagtgggcgacagatgtgaaagaaacgagttccagtgccaagacgggaaatgcatctcctacaagtgggtctgcgatggcagcgctgagtgccaggatggctctgatgagtcccaggagacgtgcttgtctgtcacctgcaaatccggggacttcagctgtgggggccgtgtcaaccgctgcattcctcagttctggaggtgcgatggccaagtggactgcgacaacggctcagacgagcaaggctgtccccccaagacgtgctcccaggacgagtttcgctgccacgatgggaagtgcatctctcggcagttcgtctgtgactcagaccgggactgcttggacggctcagacgaggcctcctgcccggtgctcacctgtggtcccgccagcttccagtgcaacagctccacctgcatcccccagctgtgggcctgcgacaacgaccccgactgcgaagatggctcggatgagtggccgcagcgctgtaggggtctttacgtgttccaaggggacagtagcccctgctcggccttcgagttccactgcctaagtggcgagtgcatccactccagctggcgctgtgatggtggccccgactgcaaggacaaatctgacgaggaaaactgcgctgtggccacctgtcgccctgacgaattccagtgctctgatggaaactgcatccatggcagccggcagtgtgaccgggaatatgactgcaaggacatgagcgatgaagttggctgcgttaatgtgacactctgcgagggacccaacaagttcaagtgtcacagcggcgaatgcatcaccctggacaaagtctgcaacatggctagagactgccgggactggtcagatgaacccatcaaagagtgcgggaccaacgaatgcttggacaacaacggcggctgttcccacgtctgcaatgaccttaagatcggctacgagtgcctgtgccccgacggcttccagctggtggcccagcgaagatgcgaagattga